(19) United States
(12) Patent Application Publication
(10) Pub. No.: US 2020/0003760 A1
(43) Pub. Date: Jan. 2, 2020

(54) IN-VITRO METHODS FOR CLASSIFYING DRUGS ACCORDING TO THEIR POTENTIAL TO CAUSE LIVER CELL INJURY

(71) Applicant: Board of Trustees of Michigan State University, East Lansing, MI (US)

(72) Inventors: Robert A. ROTH, Okemos, MI (US); Patricia E. Ganey, Okemos, MI (US); Sarah L. Hession, Holt, MI (US)

(21) Appl. No.: 16/456,972

(22) Filed: Jun. 28, 2019

Related U.S. Application Data
(60) Provisional application No. 62/809,133, filed on Feb. 22, 2019.
(60) Provisional application No. 62/692,360, filed on Jun. 29, 2018.

(51) Int. Cl. G01N 33/50; G16B 5/00

(52) U.S. Cl. CPC G01N 33/5067; G16B 5/00; G01N 33/5014

(57) ABSTRACT

The invention provides in vitro methods for classifying drugs according to their potential to cause liver injury

### RELATED APPLICATIONS

This application claims priority to U.S. Provisional Application 62/692,360 filed on Jun. 29, 2018, and U.S. Provisional Application 62/809,133 filed on Feb. 22, 2019, each of which are hereby incorporated by reference in their entirety.

### GOVERNMENT INTEREST

This invention was made with government support under DK061315 and DK112695 awarded by the National Institutes of Health. The government has certain rights in the invention.

### BACKGROUND OF THE INVENTION

Idiosyncratic, drug-induced liver injury (IDILI) is a typically rare reaction that occurs at drug doses that are safe in the majority of patients. Cases of IDILI can be severe, leading to liver transplantation or death (Ostapowicz et al. 2002). In addition to public health concerns, IDILI is a common cause of removal of drugs from the pharmaceutical market due to the occurrence and severity of these reactions and to the poor ability of standard toxicity tests to identify drug candidates with IDILI liability before they reach the market (Watkins 2005, Aithal et al. 2011). The causes of IDILI are unknown, but it is thought that genetic and/or environmental factors predispose patients to toxicity from an otherwise safe dose of a drug (Roth and Ganey 2011). Because these reactions are usually rare, drugs with IDILI potential are often not identified during clinical trials that employ limited numbers of human subjects. More effective preclinical strategies to identify drug candidates with IDILI potential could inform decisions about whether to allow a drug candidate to proceed through the development process. An approach in vitro that uses cells that are readily available and easily grown in culture, requires little compound, employs a single, relevant endpoint and is amenable to high-throughput format would be highly desirable.

Development of such an approach has been challenging due to the limited knowledge about mechanisms underlying IDILI. It is commonly believed that adaptive and/or innate immune system activation underlies IDILI pathogenesis. Activation of immune cells culminates in the release of immune mediators such as cytokines. Some recently developed animal models as well as human genetic association studies suggest that adaptive immunity plays a role in the precipitation of IDILI responses to some drugs (Chakraborty et al. 2015, Lucena et al. 2011). Mice that have impaired immune tolerance developed liver injury after several administrations of IDILI-associated drugs such as halothane and amodiaquine (Chakraborty et al. 2015, Pardoll et al. 2012). Although these models involving activation of the adaptive immune system resulted in only mild liver injury, they could represent an advance in understanding IDILI pathogenesis. So far, very few animal models of IDILI have been developed that recapitulate the severity of hepatocellular injury observed in humans. Most of these are based on the interaction of drugs with an activated innate immune system (Roth and Ganey, 2011); however, a recent model demonstrated that a small proportion of mice from the Diversity Outbred panel exhibited severe hepatocellular injury in response to treatment with green tea extract (Church et al. 2015). Among the models based on the interaction between drugs and the innate immune system, the inflammatory mediators tumor necrosis factor-alpha (TNF) and/or interferon-gamma (IFN) were critical to the pathogenesis of liver injury (Dugan et al. 2011, Hassan et al. 2008, Lu et al. 2012, Shaw et al. 2009a, Shaw et al. 2009b, Zou et al. 2009).

Activation of immune cells culminates in the release of immune mediators such as cytokines. Some recently developed animal models as well as human genetic association studies suggest that adaptive immunity plays a role in the precipitation of IDILI responses to some drugs (Lucena et al., 2011; Chakraborty et al., 2015). Mice that have impaired immune tolerance developed liver injury after several administrations of IDILI-associated drugs such as halothane and amodiaquine (Metushi et al., 2015; Chakraborty et al., 2015). Although these models involving activation of the adaptive immune system resulted in only mild liver injury, they could represent an advance in understanding IDILI pathogenesis. Thus far, very few animal models of IDILI have been developed that recapitulate the severity of hepatocellular injury observed in humans. Most of these are based on the interaction of drugs with an activated innate immune system (Roth and Ganey, 2011). Among the models based on the interaction between drugs and the innate immune system, the inflammatory mediators tumor necrosis factor-α (TNF) and interferon-γ (IFN) were critical to the pathogenesis of liver injury (Hassan et al., 2008; Shaw et al., 2009a,b; Zou et al., 2009; Dugan et al., 2011; Lu et al., 2012).

Both innate and adaptive immune responses culminate in the release of these potentially cytotoxic, pro-inflammatory cytokines. Findings from the animal studies raised the possibility that IDILI-associated drugs sensitize hepatocytes to cell death signaling from cytokines such as TNF and IFN (Roth and Ganey, 2011). Indeed, using a series of drugs Cosgrove et al. (2009) found a correlation between IDILI liability and ability of drugs to synergize with cytokines to kill primary human hepatocytes in vitro. Using a smaller subset of drugs, they also found that their results in primary human hepatocytes could be reproduced using HepG2 cells, suggesting that the latter cells hold promise in classifying drugs according to IDILI liability. These and other studies suggest that IDILI-associated drugs act in part by causing stress to hepatocytes, such that they become susceptible to killing mediated by cytokines (Beggs et al. 2014, Beggs et al. 2015, Cosgrove et al. 2009, Fredriksson et al. 2011, Fredriksson et al. 2014, Maiuri et al. 2015, Zou et al. 2009).

Using HepG2 cells, we recently studied the cytotoxic interaction of TNF/IFN with a series of nonsteroidal anti-inflammatory drugs (NSAIDs) with various IDILI liabilities and also with an antibiotic, trovafloxacin (Beggs et al. 2014, Beggs et al. 2015, Maiuri et al. 2015). In studies presented here, we expand on those findings with a larger set of drugs. Importantly, elucidation of detailed concentration-response relationships permitted calculation of various parameters (e.g. EC50, maximal response, slope, etc.) that we then incorporated into statistical models to evaluate the ability of this approach to classify drugs according to their IDILI liabilities. The results suggest a highly promising, in vitro approach to predict IDILI liability.

### SUMMARY OF THE INVENTION

The present invention provides an in vitro method of classifying a drug according to the drug's potential to cause liver cell injury, comprising:

a) obtaining a population of liver cells;

b) contacting the population of liver cells with a drug provided at a range of concentrations;

c) contacting the population of liver cells with a cytokine provided at a range of concentrations;

d) determining cytotoxicity of the population of liver cells;

e) generating a concentration-response curve;

f) defining covariates from the curve using a four-parameter logistic model;

g) developing a classification model using logistic regression of covariates defined in step h) to generate a logistic regression model; and

i) evaluating by receiver operating characteristic (ROC) the optimal classification model and covariate combination, to thereby classify the drug according to the drug's potential to cause liver injury.

In some embodiments, the liver cells are primary human heptocytes.

In some embodiments, the hepatoma cells are human hepatoma cells.

In some embodiments, the human hepatoma cells are HepG2.

In some embodiments, the liver cell injury is a hepatocellular injury.

In some embodiments, the liver cell injury is liver death.

In some embodiments, the hepatocellular injury is idiosyncratic, drug-induced liver injury (IDILI).

In some embodiments, the drug is selected from the group consisting of steroidal or nonsteroidal anti-inflammatory drugs (NSAIDs), antibiotic, anti-viral, anti-bacterial, anti-fungal, chemotherapeutic, small molecule drugs of any pharmacologic class, cardiac, pulmonary, lipid-modulating, neuromodulatory, analgesic, drugs that modify blood coagulation, gastrointestinal (GI) drugs, anti-convulsants, and endocrine drugs.

In some embodiments, the drug is selected from any one of the drugs set forth in Table 1.

In some embodiments, the cytokine is selected from the group consisting of IL-3, IL-4, tumor necrosis factor-alpha (TNF-α), TNF-β, LT-β, interleukin-2 (IL-2), IL-7, IL-9, IL-15, IL-13, IL-5, IL-1α, IL-1β, interferon-gamma (IFN-γ), IL-10, IL-17, IL-16, IL-18, HGF, IL-11, MSP, FasL, TRAIL, TRANCE, TWEAK, CD27L, CD30L, CD40L, APRIL, TALL-1, 4-1BBL, OX40L, GITRL, IGF-1, IGF-II, MSP, FGF-α, FGF-β, FGF-3-19, NGF, BDNF, NTs, Tpo, Epo, Ang1-4, PDGF-AA, PDGF-BB, VEGF-A, VEGF-B, VEGF-C, VEGF-D, PIGF, EGF, TGF-α, AR, BTC, HRGs, HG-EGF, SMDF, OB, CT-1, CNTF, OSM, MK, and PTN.

In some embodiments, the cytokine is TNF-α, IFN-γ, or both.

In some embodiments, the cytotoxicity is measured as lactate dehydrogenase (LDH) activity released from cells.

In some embodiments, the cytotoxicity is measured as percent (LDH) activity released from cells.

In some embodiments, the cytotoxicity is measured as percent cellular ATP released from cells.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 show depiction of base covariates considered for evaluation. Blue curve represents hypothetical response to drug alone; red curve represents response to drug in the presence of cytokine. Min=LDH release in the absence of drug. Max=maximal LDH release. Delta=max(LDH)−min(LDH) for each curve. Although not depicted in the figure, Deltadiff=Delta cytokine−DeltaVEH. EC50 is the [drug]/Cmax value at 50% Delta. R10 is not depicted but it represents the [drug]/Cmax value associated with a 10% increase in LDH release above min(LDH).

FIG. 2 depicts drug/cytokine-induced cytotoxicity: concentration-response. HepG2 cells were treated with 14 drugs associated with IDILI and 10 drugs not associated with IDILI, alone (VEH) and in combination with TNF and/or IFN. Cytotoxicity (% LDH release) was evaluated 24 hours after treatment. The numbers listed on the x-axis represent the concentration of drug relative to Cmax (fold Cmax). Refer to Table 1 for the Cmax information and IDILI classification for each drug and to the methods section for the rationale concerning the range of drug concentrations evaluated. Each data point represents the mean±standard error of the mean (S.E.M.) of at least 3 separate experiments. The dotted curves indicate that the treatment condition (i.e. VEH, TNF, IFN or TNF/IFN) resulted in a statistically significant change in LDH from baseline (no drug) at one or more drug concentrations (ANOVA p<0.01). Solid lines indicate that the treatment condition did not result in a statistically significant change in LDH relative to baseline at any drug concentration (ANOVA p≥0.01). The dotted curves were modeled using a four-parameter logistic function as described in Methods in order to compute parameters describing curve characteristics (minimum cytotoxic effect, maximum cytotoxic effect, EC50, etc.).

FIG. 3 contains two panels, Panels (A) and (B), depicting the comparison of the model incorporating daily dose to that incorporating Cmax. Panel (A) shows AUCs and 95% confidence intervals are depicted for the ROC curves derived from the models incorporating either daily dose or Cmax. The covariates are listed on the left, and the AUC for each one is shown on the right next to the 95% confidence interval, which is shown in brackets. Panel (B) shows the ROC curves for the model incorporating daily dose and Cmax are indicated by red line and blue lines, respectively. The 95% confidence intervals for the model incorporating daily dose and Cmax are shaded red or blue, respectively. Overlap between the confidence intervals for the two ROC curves appears violet.

FIG. 4 contains two panels, Panels (A) and (B), depicting evaluation of models incorporating the base covariates. Panel (A) shows AUCs and 95% confidence intervals are illustrated for the ROC curves derived from the models incorporating the base covariates Delta VEH, Delta TNF, EC50 VEH, EC50 TNF, EC10 VEH, EC10 TNF, R10 VEH or R10 TNF. The covariates are listed on the left, and the AUC for each is shown on the right next to the 95% confidence interval, which is in brackets. *denotes a statistically significant difference as determined by DeLong's test (p<0.05). Panel (B) shows ROC curves were generated and indicate for each model the 95% confidence interval shaded in grey. The covariates incorporated in the model are listed on the bottom right corner of each ROC curve.

FIG. 5 contains two panels, Panels (A) and (B), depicting evaluation of models incorporating the derived covariates. Panel (A) shows AUCs and 95% confidence intervals for the ROC curves are depicted for the models incorporating the derived covariates individually. The covariates are listed on the left, and the AUC for each is shown on the right next to the 95% confidence interval, which is in brackets. Panel (B) shows ROC curves were generated and indicate for each model the 95% confidence interval shaded in grey. The covariate incorporated in the model is listed on the bottom right corner of each ROC curve.

FIG. 6 shows evaluation of models incorporating combinations of the base and derived covariates with and without Cmax. AUCs and 95% confidence intervals for the ROC curves are depicted for the models incorporating various combinations of base and derived covariates in the absence (upper half) and presence (lower half) of Cmax. The covariates are listed on the left, and the AUC for each is shown on the right next to the 95% confidence interval, which is in brackets.

FIG. 7 shows ROC curves with an AUC=0.95. ROC curves for which AUC 0.95 are depicted. The 95% confidence interval is shaded grey. The covariates incorporated into the model are listed at the bottom right corner of each ROC curve. The ROC curves shown were not significantly different from each other as determined by DeLong's test (p>0.05).

FIG. 8 shows models incorporating the covariate TNF change. AUCs and 95% confidence intervals for the ROC curves are depicted for the models incorporating TNF change with other covariates in the absence (upper panel) and presence (lower panel) of Cmax. The covariates are listed on the left, and the AUC for each is shown on the right next to the 95% confidence interval, which is in brackets.

FIG. 9 contains two panels, Panels (A) and (B), depicting comparison of the model incorporating Cmax from a set of 24 drugs to the model incorporating Cmax from a set of 272 drugs. Panel (A) shows AUCs and 95% confidence intervals are depicted for the ROC curves derived from the models incorporating either Cmax from the set of 24 drugs used in these studies or Cmax from a set of 272 drugs. The covariates are listed on the left, and the AUC is shown on the right next to the 95% confidence interval, which is in brackets. Panel (B) shows the ROC curves for the set of 24 drugs and the set of 272 drugs are indicated by the black line and red line, respectively. The 95% confidence intervals for the model describing the set of 24 drugs and for the model describing the set of 272 drugs are shaded grey and red, respectively.

FIG. 10 contains three panels, Panels (A)-(C), depicting comparison of models incorporating covariate(s) that describe the drug/TNF concentration response curve to those that include response to IFN. Panel (A) shows AUCs and 95% confidence intervals are shown for the models containing the individual covariates Delta TNF, Delta TNF/IFN, EC50 TNF, EC50 TNF/IFN, EC10 TNF, EC10 IFN, R10 TNF and R10 TNF/IFN and B) the models combining the covariates Delta TNF and EC50 TNF or Delta TNF, EC50 TNF, Delta TNF/IFN and EC50 TNF/IFN. For Panels (A) and (B), the covariates are listed on the left, and the corresponding AUCs are shown on the right next to the 95% confidence intervals, which are in brackets. Panel (C) shows ROC curves are shown for the two models listed in B) and indicate the 95% confidence interval, shaded in grey. The covariates incorporated into the model are listed at the bottom right corner of each ROC curve.

FIG. 11 shows drug-cytokine concentration response curves. HepG2 cells were treated with 14 drugs associated with IDILI and 10 drugs not associated with IDILI, alone (VEH) and in combination with TNF and/or IFN. Cytotoxicity (% LDH release) was evaluated 24 hours after treatment. The numbers listed on the x-axis represent the concentration of drug relative to Cmax (fold Cmax). The data presented are the same as in FIG. 2; however, the axes have been adjusted such that the y-axis ranges from 0-100% LDH Release and the x-axis ranges from 0-100 X Cmax (or greater than 100 X Cmax in cases where higher concentrations were tested).

FIG. 12 shows cytotoxic diclofenac-TNF interation in high throughput format. HepG2 cells were plated in 384-well plates (5000 cells/well) and treated with TNF (10 ng/ml) and diclofenac in the ADDRC facility as described in Subaim A. N=1 (3 replicates in the same plate).

FIG. 13 shows 2-Hydroxyflutamide-TNF concentration response curve. HepG2 cells were treated with 2-OH-flutamide alone (Vehicle) and in combination with TNF. Cytotoxicity (% LDH release) was evaluated 24 hours after treatment. The numbers listed on the x-axis represent the concentration of drug relative to Cmax (fold Cmax). The dotted curves indicate that the treatment condition (i.e. VEH, TNF, IFN or TNF/IFN) resulted in a statistically significant change in LDH from baseline (no drug) at one or more drug concentrations (ANOVA p<0.01).

FIG. 14 contains two panels, A and B, and shows graphs of complete CRRs (shown as points) for trovafloxacin alone (A) or in the presence of TNF (B) and fitted 4-parameter log-logistic CRC models (shown as lines). For this study, CRRs were censored above their respective EC50s to simulate incomplete CRRs.

FIG. 15 shows graphs of nine CRC models fit to the censored CRRs for trovafloxacin+TNF as an example. AIC values, measures of model fit, are shown for each model. Lower AIC values indicate better fit. The CRC model selected for estimation of covariates is circled in red. LL.4=4-parameter log-logistic model, AR.3=3-parameter asymptotic regression model, EXD.3=3-parameter exponential model, W1.3&W2.3=3-parameter Weibull models, W1.4&W2.4=4-parameter Weibull models, G.3=3-parameter Gompertz model, G.4=4-parameter Gompertz model.

FIG. 16 contains two panels, A and B, and contains a 4-parameter log-logistic CRC models fit to the censored CRRs (low-concentration region) for trovafloxacin alone (A) and in the presence of TNF (B). The horizontal blue line illustrates the mean control value estimated by pooling LDH % responses at the zero concentrations for both CRRs above. The horizontal dashed red line illustrates an upper limit of the pooled control data as the control mean plus 2 control standard deviations. PODs were then calculated for each curve as the effective concentration corresponding to the upper limit of the pooled control data, shown as vertical dashed red lines.

FIG. 17 provides a comparison of the classification accuracy for each model. The model including both POD.DRUG and POD.DRUG+TNF as covariates yielded the best classification accuracy with an AUC of 0.95.

FIG. 18 shows an ROC Curve for the best classification model.

FIG. 19 shows predicted probability of drug being IDILI+ for each drug according to the five models.

Note that for every figure containing a histogram, the bars from left to right for each discreet measurement correspond to the figure boxes from top to bottom in the figure legend as indicated.

### DETAILED DESCRIPTION OF THE INVENTION

Idiosyncratic, drug-induced liver injury (IDILI) typically occurs in a small fraction of patients and has resulted in removal of otherwise efficacious drugs from the market. Current preclinical testing methods are ineffective in predicting which drug candidates have IDILI liability. Recent results suggest that immune mediators such as tumor necrosis factor-alpha (TNF) and interferon-gamma (IFN) interact with drugs that cause IDILI to kill hepatocytes. The purpose of this study was to test the hypothesis that the ability of a drug to synergize with these inflammatory cytokines to cause hepatocellular death in vitro can classify drugs according to their potential to cause IDILI in humans. Human hepatoma (HepG2) cells were treated with drugs associated with IDILI or with drugs lacking IDILI liability and cotreated with TNF and/or IFN. Out of 14 drugs associated with IDILI, almost all synergized with TNF to kill HepG2 cells. IFN enhanced the toxicity mediated by some IDILI-associated drugs in the presence of TNF. In contrast, of 10 drugs with little/no IDILI liability, none synergized with inflammatory cytokines to kill HepG2 cells. Detailed concentration-response relationships were determined for calculation of parameters such as the maximal cytotoxic effect, slope and EC50 for use as covariates for classification modeling using logistic regression. These parameters were incorporated into multiple classification models to identify combinations of covariates that most accurately classified the drugs according to their association with human IDILI, resulting in an optimal model that classified the drugs according to their IDILI liability with extraordinary selectivity and specificity.

The purpose of this study was to develop and evaluate an in vitro approach to classify drugs according to their potential to cause IDILI. The overall hypothesis tested was that the ability of a drug to synergize with the cytokines TNF and/or IFN to kill HepG2 cells is associated with the drug's propensity to cause IDILI in humans. Detailed concentration response curves were generated, and this proved to be critical for development of a model with the capacity to classify drugs correctly.

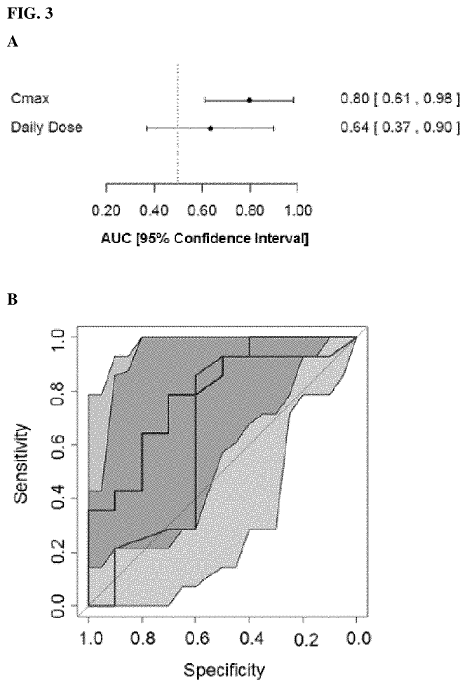

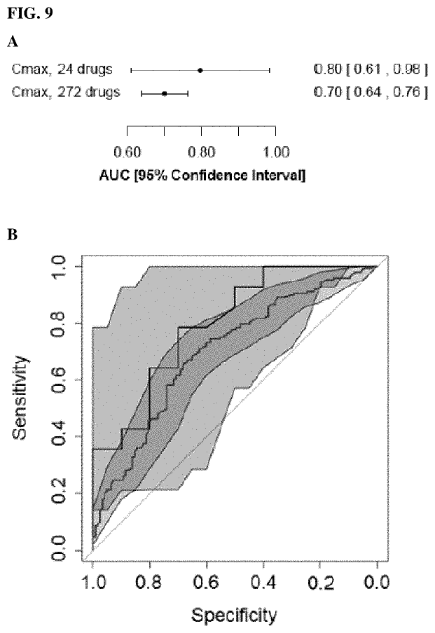

Since it has been suggested that the daily dose of a drug might be associated with its potential to cause IDILI and since dose is often related to Cmax, we evaluated how well daily dose or Cmax classifies drugs according to their IDILI liability. Daily dose was not effective at classifying the set of 24 drugs according to their potential to cause IDILI (FIG. 3). Interestingly, Cmax was somewhat effective at classifying the set of 24 drugs, suggesting that plasma drug concentration is a contributor to IDILI. However, it is clear that the use of Cmax as the sole covariate did not lead to a model with great classification ability (FIGS. 9A and 9B).

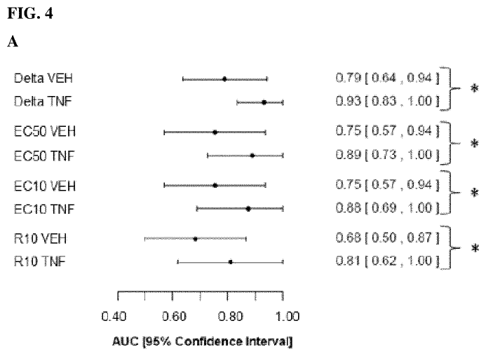

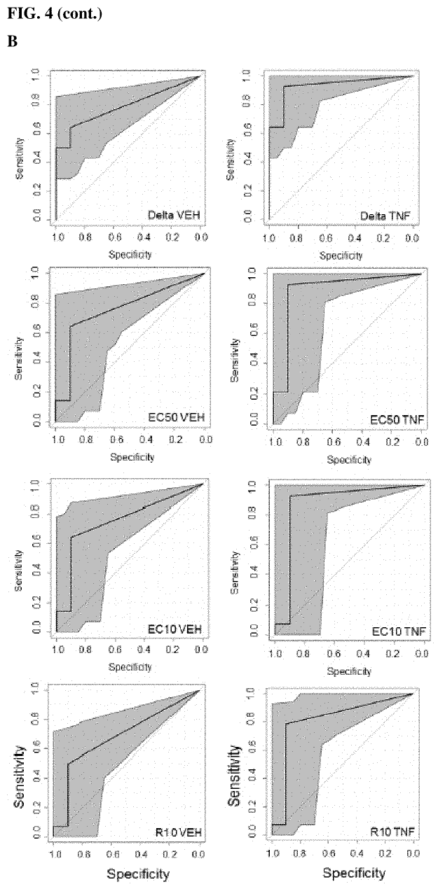

We then determined whether cytotoxicity induced by treatment with drugs in the absence of cytokines could produce a high performing model. Models employing only individual base covariates describing cytotoxicity in the absence of TNF performed no better than Cmax (compare FIGS. 3 and 4). In contrast, the models incorporating TNF performed significantly better in classifying the drugs.

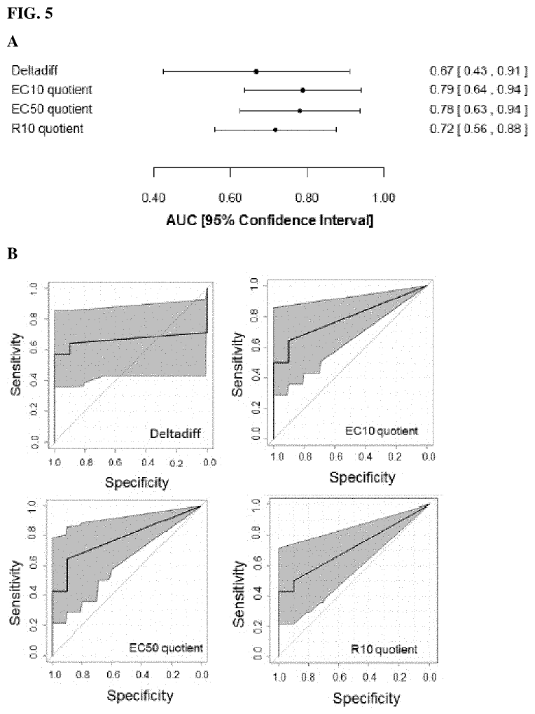

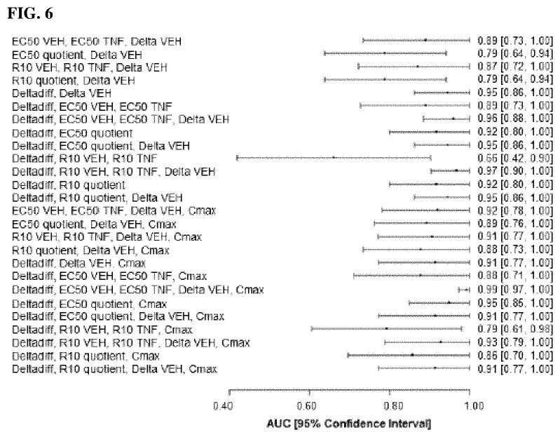

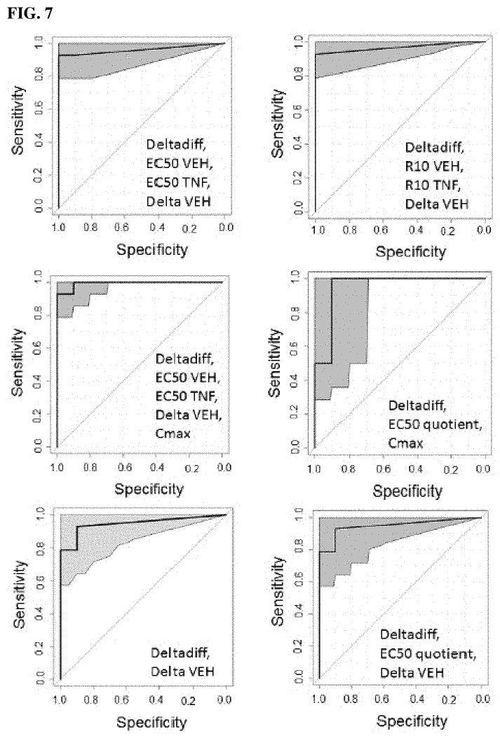

The derived covariates, when evaluated individually, did not produce more desirable ROC curves than the base covariates (compare FIG. 5 with FIG. 4). However, when covariates that account for TNF-induced changes in potency and/or efficacy were combined with those derived from drug alone, much better models resulted (FIG. 6, FIG. 7). Furthermore, incorporating Cmax into these models led to the ROC curve with the greatest AUC (0.99) and narrowest confidence interval [0.97, 1] (FIG. 6, FIG. 7). The coefficients and test statistic values for the best performing classification model, which incorporated Deltadiff, EC50VEH, EC50TNF, DeltaVEH and Cmax as covariates, are listed in Table 2. A cutoff value is an estimated probability above which a drug would be classified as associated with IDILI (1=associated with IDILI) and below which a drug would be classified as not associated with IDILI (0=not associated with IDILI). The optimal cutoff threshold is the probability cutoff that permits the most accurate classification of drugs according to IDILI liability for a given model, i.e., the point on the ROC curve closest to the coordinate (1,1). Table 3 shows the sensitivity and specificity of the best performing model when the optimal cutoff threshold was applied. Based on this model, the estimated probability that a specific drug from the set of 24 drugs is associated with IDILI is shown in Table 4. As can be seen in the Table, this classification model led to almost complete separation between IDILI-associated drugs and drugs that are not associated with IDILI. If this model were to be used in a preclinical safety evaluation setting to predict IDILI potential of a set of drugs, the user could either select the optimal cutoff threshold or choose a cutoff threshold that is either more or less sensitive depending on what false positive rate is deemed acceptable.

TABLE 2
Logistic regression coefficients and p-values for the optimal
classification model incorporating the covariates Deltadiff,
EC50 VEH, EC50 TNF, Delta VEH and Cmax. The coefficients
(beta values) were computed using Firth's approach as
described in Methods. A p-value < 0.05 indicates that the
covariate contributes significantly to the prediction of outcome
(IDILI liability).

| Covariates | Beta | Chi square | p-value |
|---|---|---|---|
| Intercept | −1.924 | 7.091 | 0.008 |
| Deltadiff | 0.108 | 2.471 | 0.116 |
| EC50VEH | −0.066 | 6.215 | 0.013 |
| EC50TNF | 0.050 | 4.480 | 0.034 |
| DeltaVEH | 0.081 | 11.038 | 0.001 |
| Cmax | 0.0031 | 0.129 | 0.720 |

TABLE 3
Sensitivity and specificity for the optimal classification model
incorporating the covariates Deltadiff, EC50 VEH, EC50 TNF,
Delta VEH and Cmax. The optimal cutoff threshold (k*) is shown
as is the specificity and sensitivity of the model at k*. Also
indicated are the area under the ROC curve (AUC) and the
95% confidence intervals for the specificity, sensitivity and AUC.

| | | 95% confidence interval |
|---|---|---|
| Optimal cutoff threshold (k*) | 0.46 | |
| True negative rate (specificity) | 1 | (0.7, 1) |
| using threshold k* | | |
| True positive rate (sensitivity) | 0.93 | (0.79, 1) |
| using threshold k* | | |
| AUC | 0.99 | (0.97, 1) |

TABLE 4
Estimated probabilities that a drug is associated with IDILI
computed from the best performing logistic regression model
employing Deltadiff, EC50 VEH, EC50 TNF, Delta VEH and
Cmax as covariates. With regard to the true IDILI classification
of drugs, IDILI(−) indicates that the drug is not associated with
IDILI and IDILI(+) indicates that the drug is associated with
IDILI in human patients.

| | Estimated | True |
|---|---|---|
| Drug | probability | classification |
| Buspirone | 0.1275 | IDILI− |
| Idarubicin | 0.1275 | IDILI− |
| Promethazine | 0.1275 | IDILI− |
| Sertraline | 0.1275 | IDILI− |
| Azithromycin | 0.1276 | IDILI− |
| Rofecoxib | 0.1278 | IDILI− |
| Moxifloxacin | 0.1296 | IDILI− |
| Levofloxacin | 0.1329 | IDILI− |
| Aspirin | 0.1445 | IDILI− |
| Flucloxacillin | 0.1545 | IDILI+ |
| Pioglitazone | 0.2935 | IDILI− |
| Telithromycin | 0.6685 | IDILI+ |
| Flutamide | 0.7036 | IDILI+ |
| Trovafloxacin | 0.7448 | IDILI+ |
| Isoniazid | 0.7562 | IDILI+ |
| Diclofenac | 0.8578 | IDILI+ |
| Naproxen | 0.8589 | IDILI+ |
| Doxorubicin | 0.8839 | IDILI+ |
| Bromfenac | 0.9397 | IDILI+ |
| Clavulanate | 0.9509 | IDILI+ |
| Chlorpromazine | 0.961 | IDILI+ |
| Ibuprofen | 0.9733 | IDILI+ |
| Valproic Acid | 0.9909 | IDILI+ |
| Nimesulide | 1 | IDILI+ |

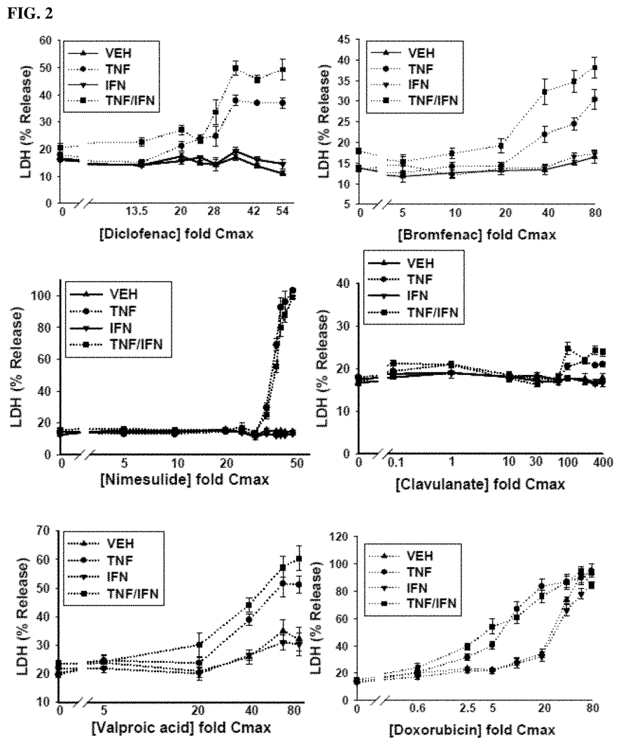

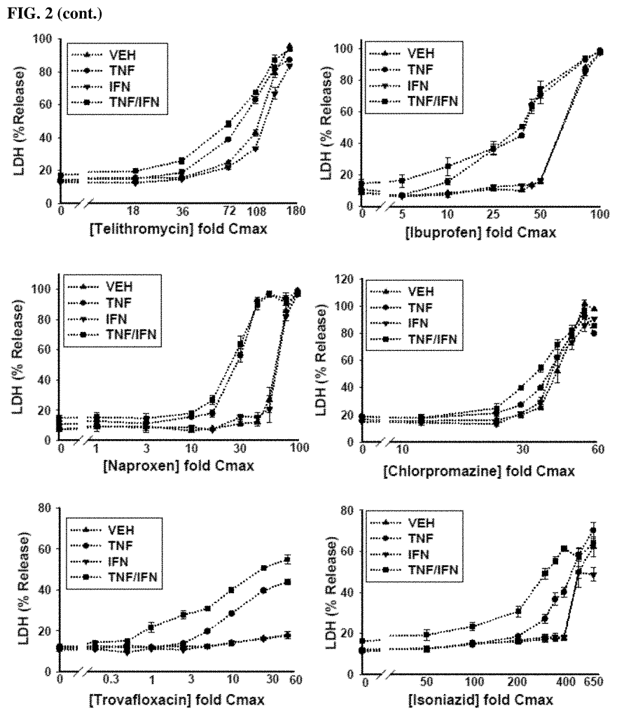

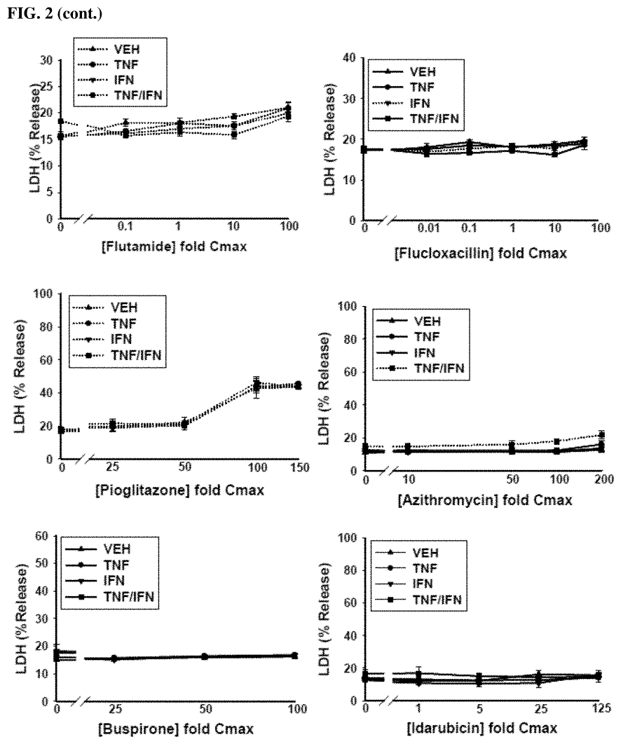

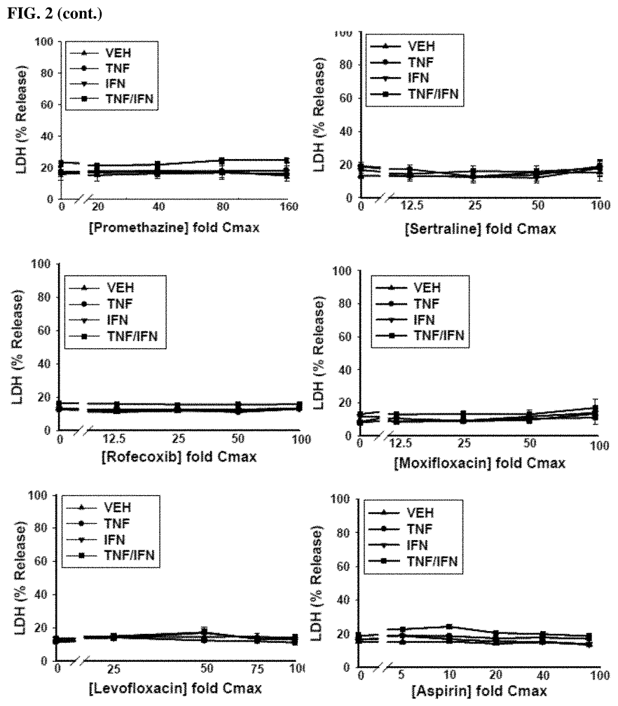

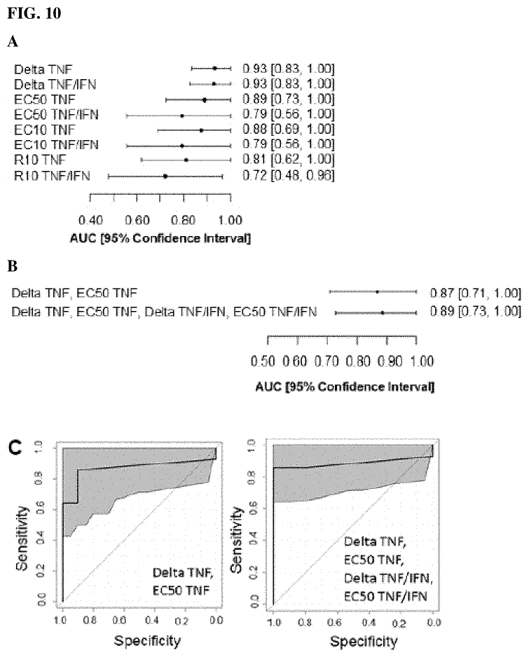

IFN contributed to hepatotoxicity in several animal models of IDILI and was therefore of interest to include in our examination (Shaw et al. 2009, Hassan et al. 2008, Dugan et al. 2011). Interestingly, in the absence of TNF, IFN did not synergize with any of the drugs in vitro to cause cell death (FIG. 2). However, IFN enhanced the cytotoxic interaction between several IDILI-associated drugs and TNF. Accordingly, we evaluated whether a change in the concentration response curves due to exposure to IFN could improve the classification of drugs. The classification model developed from the covariates that described the response to drug/TNF/IFN produced ROC curves that were not improved from those incorporating covariates that describe the response to drug/TNF (FIGS. 10A and 10B). These results indicate that cytotoxic synergy between IDILI-associated drugs and TNF is sufficient to produce a statistical model that accurately classifies drugs according to their potential to cause human IDILI, irrespective of the presence of IFN. They also suggest that the cell killing activity of IFN depends on the presence of TNF.

It is worth considering the possibility that other cytokines play a role in the pathogenesis of IDILI, and it would be interesting to examine if other cytokines could interact with drugs to cause cytotoxicity in vitro and/or synergize with TNF to enhance cytotoxicity. Cosgrove et al. (2009) performed a study examining drug/cytokine interactions in vitro and found that IL-1β in combination with TNF and IFN interacted with some drugs to cause cytotoxicity. It is unclear to what extent IL-1β contributed to this interaction. However, Shaw et al. (2009) demonstrated that IL-1β levels are elevated in mice cotreated with trovafloxacin and LPS, raising the possibility that IL-β plays a role in the hepatotoxicity observed in these mice. Whether or not the presence of IL-1β or other immune mediators would improve the ability of the models presented herein to classify drugs is unknown but worth considering in future studies.

We reported recently that IFN-mediated enhancement of NSAID/TNF-induced cytotoxicity occurs with some IDILI-associated NSAIDs but not others, and this effect was related to chemical structure and to the magnitude of clinical concern about IDILI for specific NSAIDs (Maiuri et al. 2015). Specifically, several acetic acid derivatives that are associated with IDILI of greatest clinical concern synergized with TNF to cause HepG2 cell death, and IFN enhanced this effect. In contrast, two propionic acid derivatives, which are associated with IDILI that is of less clinical concern, also synergized with TNF, but IFN was without effect. In the analysis presented here, we were seeking a binary answer—IDILI potential yes or no—and inclusion of IFN did not affect the outcome. It would be interesting if the ability of drugs to sensitize cells to the harmful effects of IFN could distinguish drugs of greater concern clinically for IDILI from those of less concern. Clearly, a larger number of drugs would need to be analyzed to evaluate this.

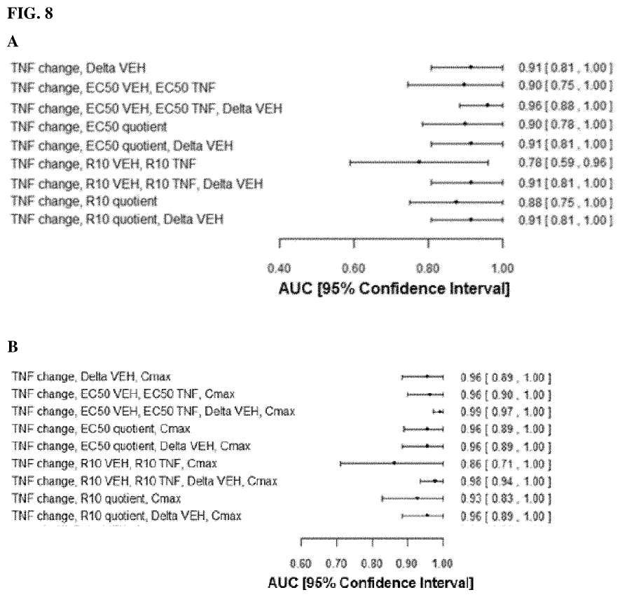

A potential challenge that might be faced when employing this assay during preclinical safety evaluation is the inability to generate complete concentration-response relationships due to solubility limitations of the drug or other factors. Computing covariates using the four-parameter logistic model requires complete concentration-response curves; however, we defined several covariates that can be computed without the need to generate a complete concentration-response curve. One of these is R10, or the drug concentration at which there is an increase of 10% LDH activity above min. Another covariate that could be computed without the need to generate a complete concentration-response curve we defined as “TNF change.” TNF change identifies those drugs only cytotoxic in the presence of TNF without the need for complete concentration-response curves. Interestingly, combining the covariates R10quotient (i.e., R10TNF/R10VEH) and TNF change resulted in an ROC curve with an AUC=0.88 and a 95% confidence interval of [0.75, 1] (FIG. 8). This suggests that a model that leads to good classification of drugs according to their potential to cause IDILI can be generated without the need to delineate complete concentration-response relationships. This model might be useful for predicting IDILI-potential of drug candidates when availability of compound is limited or when solubility limitation prevents generation of a complete concentration-response curve.

Although HepG2 cells are human-derived, their use for drug toxicity evaluation has been criticized because they have limited capacity to bioactivate drugs to toxic metabolites via cytochrome P450-mediated pathways. Despite this potential limitation, Cosgrove et al. (2009) found that HepG2 cells behave similarly to primary human hepatocytes in their cytotoxic responses to drug-cytokine combinations. We have also observed comparable responses in primary murine hepatocytes (Zou et al. 2009, Beggs et al. 2014, Maiuri et al. 2015). These findings suggest either that (1) metabolic activation of drugs by HepG2 cells, although limited, is sufficient to stress cells so that they respond to cytokine exposure by dying or (2) metabolism is not generally needed for the cytotoxic interaction of drugs with cytokines.

Visual inspection of FIG. 2 revealed three IDILH drugs for which there was modest (flutamide, clavulanate) or no (flucloxacillin) response. Despite this, statistical modeling classified flutamide and clavulanate as IDILI1. In the case of flucloxacillin, analysis of variance of the concentration response data determined that there was no statistically significant increase in LDH alone or in the presence of TNF. As a result, covariates were set to zero with the exception of Cmax, and the calculated probability for flucloxacillin was low (0.154; see the formula in the Materials and Methods). In contrast, analysis of variance applied to the concentration response data for flutamide alone and in combination with TNF detected very small but statistically significant increases in LDH release relative to baseline (FIG. 2; FIG. 8). Consequently, nonzero covariates were generated from the concentration-response data. Similarly for clavulanate, there was an interaction with TNF that was small but statistically significant, resulting in nonzero covariates. These nonzero covariates led to calculated probabilities that, at the optimal cutoff, identified flutamide and clavulanate correctly as IDILI1. The pronounced reproducibility of even small changes using HepG2 cells and the apparent sensitivity of the model in these two cases may be a strength of this approach to classification.

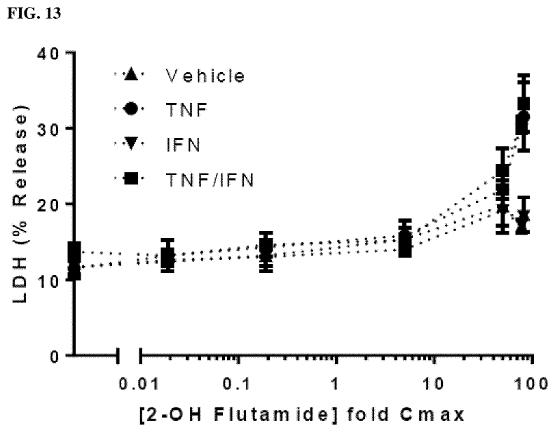

Flutamide is metabolized in vivo to 2-hydroxyflutamide (Cₘₐₓ, 5.74 μM), which is more potent pharmacologically as an antiandrogen (Brogden and Clissold, 1989) and is thought to contribute to IDILI responses (Ball et al., 2016). To strengthen this proof-of-concept study and to further evaluate the modest cytotoxic effect of flutamide, we investigated the performance of 2-hydroxyflutamide in the assay. Concentration-response curves were generated (FIG. 13), and covariates derived from the curves were used in the best-performing classification model. A high probability for association with IDILI was calculated (0.999) for 2-hydroxyflutamide. Furthermore, we replaced the flutamide covariate data in the bestperforming model with the 2-hydroxyflutamide covariates. Recalculation of the best-performing classification model yielded coefficients similar to those presented in Table 2 (Table 15), the same AUC for the ROC curve, and no change in classification of drugs as IDILI1 or IDILI2. These results strengthen the conclusions based on the modest cytotoxic response to flutamide and support the approach to classifying IDILI-associated drugs.

The observation that flucloxacillin was incorrectly classified as not associated with IDILI suggests that the bestperforming model, although seemingly promising, has limitations. It is worth noting that flucloxacillin typically produces liver injury in humans that is classified as cholestatic rather than hepatocellular (Enat et al., 1980; Williams and Malatjalian, 1981; Bengtsson et al., 1985; Moseley, 2013). Clavulanate is also associated predominately with a cholestatic pattern of injury in human patients (Sanchez-Ruiz-Granados et al., 2012; Beraldo et al., 2013) and interacted only weakly with TNF. Accordingly, it is possible that the approach described herein is more robust in classifying drugs that cause hepatocellular rather than cholestatic patterns of injury.

In summary, the results add to evidence that drug-induced stress can sensitize hepatocytes to the killing actions of cytokines such as TNF and IFN. Moreover, this could be requisite for the pathogenesis of IDILI, since numerous IDILI-associated drugs show cytotoxic synergy with cytokines in vitro at drug concentrations near those that occur in patients. Currently, effective assays to screen preclinically for IDILI potential are lacking. A method that accurately identifies drug candidates with the potential to cause IDILI could revolutionize preclinical testing strategies. Our results suggest an in vitro assay that could do just that, i.e., by delineating drug concentration-response curves in the absence and presence of TNF and employing resulting covariates in an appropriate statistical model for classification. One of the strengths of this approach is that the user would have discretion to choose a level of risk tolerance guided by the results of ROC analysis. That choice could depend on a variety of factors, including risk tolerance in the context of the therapeutic use, other drug candidates that are in contention for going forward into development, etc. For example, if several potentially effective compounds with no apparent toxicity were identified in early preclinical screens but some returned an “IDILI+” result in a drug-cytokine assay, this might prompt a decision to pursue other candidates for development. The magnitude of interaction with TNF assessed by direct inspection of concentration-response curves (FIG. 2) might also be used to inform such decisions. Overall, this classification approach is attractive because it (1) uses a cell type that is easily obtained and maintained in culture and yields consistent results, (2) requires minimal amounts of test compound, (3) employs a single, easily and inexpensively measured phenotypic endpoint that is directly relevant to IDILI (hepatocellular death), (4) is based on interaction between drug and a product of immune system activation likely to be relevant to IDILI pathogenesis and (5) is adaptable to high throughput technology. Validation of this approach as a screening tool will require the evaluation of additional drugs, but the results presented herein seem quite promising.

As can be appreciated from the disclosure above, the present invention has a wide variety of applications. The invention is further illustrated by the following examples, which are only illustrative and are not intended to limit the definition and scope of the invention in any way.

### EXAMPLES

### Example 1: Materials and Methods

### Materials

All drugs were purchased from Sigma-Aldrich (St. Louis, Mo.) or Santa Cruz Biotechnology (Dallas, Tex.) unless otherwise noted. Recombinant human TNF and IFN were purchased from R & D Systems (Minneapolis, Minn.) or Millipore (Billerica, Mass.). Phosphate-buffered saline (PBS), Dulbecco's Modified Eagles Medium (DMEM), fetal bovine serum (FBS), Antibiotic-Antimycotic (ABAM) and 0.25% Trypsin-EDTA were purchased from Life Technologies (Carlsbad, Calif.).

### Cell Culture

Human hepatoma HepG2 cells (American Type Culture Collection, Manassas, Va.) were grown in 25-cm² tissue culture flasks, maintained in DMEM supplemented with 10% FBS and 1% ABAM in a humidified incubator at 37° C. under 95% air and 5% CO₂. Cells were passed or used for experiments when they reached approximately 80% confluence. Cells were used at passage 6-16.

### IDILI Classification

The set of 24 drugs evaluated in this study were classified as being associated with (IDILI+) or not associated with IDILI (IDILI−). Classification was based on a set of criteria established by Xu et al. (2008). Table 1 lists the drugs evaluated in this study, their maximal plasma concentration (Cmax) after pharmacologic dosing in human patients and their IDILI classification.

TABLE 1
IDILI classification, daily dose, Cmax and references from which
the Cmax values were taken. IDILI classification was determined
by a set of criteria described in Xu et al. (2008). IDILI(−)
indicates that the drug is not associated with IDILI, whereas
IDILI(+) indicates that the drug is associated with IDILI.

| | IDILI | Cmax | Daily dose | Cmax |
|---|---|---|---|---|
| Drug | liability | (μM) | (mg) | Reference |
| Aspirin | IDILI− | 47 | 1300 | Brandon et al. |
| | | | | 1986 |
| Azithromycin | IDILI− | 0.5 | 500 | Xu et al. 2008 |
| Buspirone | IDILI− | 0.005 | 15 | Xu et al. 2008 |
| Idarubicin | IDILI− | 0.02 | 1 | Xu et al. 2008 |
| Levofloxacin | IDILI− | 15.7 | 500 | Xu et al. 2008 |
| Moxifloxacin | IDILI− | 6.2 | 400 | Stass et al. 1998 |
| Pioglitazone | IDILI− | 2.67 | 15 | Xu et al. 2008 |
| Promethazine | IDILI− | 0.06 | 25 | Xu et al. 2008 |
| Rofecoxib | IDILI− | 1 | 12.5 | Gottesdeiner et |
| | | | | al. 2003 |
| Sertraline | IDILI− | 0.06 | 50 | Xu et al. 2008 |
| Bromfenac | IDILI+ | 13.5 | 50 | Gumbhir-Shah et |
| | | | | al. 1997 |
| Chlorpromazine | IDILI+ | 0.84 | 200 | Xu et al. 2008 |
| Clavulanate | IDILI+ | 12 | 125 | Hu et al. 2002 |
| Diclofenac | IDILI+ | 7.44 | 100 | Xu et al. 2008 |
| Doxorubicin | IDILI+ | 1 | 1 | Barpe et al. 2010 |
| Flucloxacillin | IDILI+ | 72.6 | 250 | Roder et al. 1995 |
| Flutamide | IDILI+ | 0.36 | 750 | Xu et al. 2008 |
| Ibuprofen | IDILI+ | 164 | 800 | Bramlage et al. |
| | | | | 2008 |
| Isoniazid | IDILI+ | 77 | 300 | Xu et al. 2008 |
| Naproxen | IDILI+ | 300 | 500 | Setiawati et al. |
| | | | | 2009 |
| Nimesulide | IDILI+ | 21.08 | 200 | Xu et al. 2008 |
| Telithromycin | IDILI+ | 2.77 | 800 | Xu et al. 2008 |
| Trovafloxacin | IDILI+ | 5 | 300 | Xu et al. 2008 |
| Valproic Acid | IDILI+ | 175 | 60 | Rha et al. 1993 |

### Cytotoxicity Assessment

HepG2 cells were plated at a density of 4×10⁴ cells per well in black-walled, 96-well, tissue culture plates and were allowed to attach overnight before being treated with compounds. Drugs were reconstituted in vehicles consisting of sterile water or DMSO (concentration less than 0.5%). Cells were treated with various concentrations of the drug or its vehicle (control) alone or in combination with the cytokines TNF (10 ng/ml) and/or IFN (10 ng/ml) or their PBS vehicle (VEH). Cytotoxicity was evaluated as lactate dehydrogenase (LDH) activity released from the cells into culture medium using the Homogeneous Membrane Integrity Assay kit from Promega (Madison, Wis.). A spectrophotometric method was used to measure percent LDH release in cases in which the drug interfered with the fluorescence-based assay, (Vanderlinde, 1985).

Concentration-response curves were generated for 24 drugs, 14 of which are associated with human IDILI and 10 of which are not. Cells were treated with drug concentrations generally ranging from 0 to 100 times the Cmax observed in human patients. This range of concentrations is based on scaling factors described in Xu et al., (2008) and accounts for variability in Cmax as well as exposure of the liver to greater concentrations. The cytokine concentrations used in this study are within 10-fold of the concentrations found in serum of human patients undergoing an inflammatory response (Pinsky et al. 1993, Taudorf et al. 2007). If a cytotoxic response was observed but did not reach a plateau by the 100 X Cmax concentration, further testing was performed with larger concentrations of drug to generate a complete (sigmoidal) concentration-response curve. Typically, the range of drug concentrations included at least two that were without effect, two defining the maximal effect and two surrounding the EC50. This was necessary because four-parameter logistic modeling used in the statistical analysis requires well defined, sigmoidal concentration-response curves. Cells were exposed to drug/cytokine combinations for 24 hours. For analysis of drug concentration-response data, concentrations of each drug were expressed as a fraction of its Cmax.

### Statistical Analysis

The statistical approach used in this study can be divided into three phases: 1) drug concentration-response determination and covariate development using four-parameter logistic models, 2) classification model development using logistic regression models, and 3) analysis of classification accuracy with receiver operating characteristic (ROC) curves.

Defining covariates: four-parameter logistic model. In the first phase, variables (potential covariates) for use in the classification analysis were defined from the analysis of drug concentration-response data. Initially, a one-way analysis of variance (ANOVA) was used as an omnibus test to determine if a particular treatment (e.g. drug alone or in combination with TNF and/or IFN) caused a significant change in LDH release relative to baseline (i.e., LDH release in the absence of drug, hereafter designated “min”). The criterion for significance for the ANOVA was set at α=0.01. A 1% level of significance was used to rule out more vigorously marginal relationships between concentration and response. For treatments that did not result in a significant change in LDH above min (p>0.01), the following was assumed for the purpose of modeling: the minimum LDH response (min)=the maximum LDH response (max). For drug/cytokine treatment combinations that did result in a statistically significant LDH response, the concentration-response data were modeled using a four parameter logistic function:

$$LDH ( x ) = min + \frac{max - min}{1 + {( x / EC 50 )}^{slope}}$$

where LDH(x) is the percentage of LDH released at a given concentration x; x=[drug]/Cmax; min=the % LDH release at 0 drug concentration (i.e., baseline); and max=the maximal LDH response (i.e., maximum % LDH release). From this equation, the drug concentration associated with 50% maximal response (EC50) and the slope of the concentration-response curve were calculated. The four-parameter logistic models were generated using R statistical software (R package “drc”) (R Core Team, 2015, Ritz and Streibig, 2005).

In addition to slope and EC50, several other “base covariates” were calculated from the concentration-response curves for use in further analyses. These were calculated for each of the 96 drug/cytokine treatment combinations evaluated in this study (24 drugs X 4 cytokine combinations) (Tables 5-13).

TABLE 5
Minimum (min) LDH percentage values. These values were
determined by the four-parameter logistic equation as described
in Methods.

| Drug | Min | min TNF | min IFN | min TNF/IFN |
|---|---|---|---|---|
| Aspirin | 11.0 | 11.0 | 15.2 | 21.9 |
| Azithromycin | 11.6 | 13.0 | 12.3 | 14.8 |
| Buspirone | 17.6 | 18.6 | 18.5 | 17.9 |
| Idarubicin | 13.9 | 13.3 | 12.2 | 15.6 |
| Levofloxacin | 14.0 | 12.1 | 13.7 | 14.5 |
| Moxifloxacin | 9.7 | 9.8 | 11.3 | 14.0 |
| Pioglitazone | 19.7 | 20.4 | 19.9 | 20.8 |
| Promethazine | 17.0 | 19.4 | 17.2 | 22.1 |
| Rofecoxib | 12.2 | 12.2 | 12.9 | 15.9 |
| Sertraline | 14.0 | 16.4 | 14.5 | 16.6 |
| Bromfenac | 13.8 | 13.4 | 13.5 | 16.3 |
| Chlorpromazine | 16.0 | 18.1 | 14.4 | 17.2 |
| Clavulanate | 17.4 | 18.1 | 17.3 | 17.4 |
| Diclofenac | 15.6 | 18.0 | 16.2 | 22.5 |
| Doxorubicin | 20.1 | 16.3 | 16.4 | 15.1 |
| Flucloxacillin | 17.9 | 17.5 | 17.0 | 16.9 |
| Flutamide | 15.7 | 15.7 | 15.8 | 16.7 |
| Ibuprofen | 8.8 | 8.9 | 9.0 | 15.8 |
| Isoniazid | 15.7 | 13.3 | 16.0 | 20.8 |
| Naproxen | 8.7 | 12.6 | 10.8 | 15.7 |
| Nimesulide | 14.6 | 14.2 | 13.1 | 16.0 |
| Telithromycin | 15.1 | 14.3 | 13.6 | 17.4 |
| Trovafloxacin | 12.1 | 11.6 | 10.6 | 12.5 |
| Valproic Acid | 17.9 | 20.4 | 19.2 | 22.5 |

TABLE 6
Maximum (max) LDH percentage values. These values were
determined by the four-parameter logistic equation as described
in Methods

| Drug | max | max TNF | max IFN | max TNF/IFN |
|---|---|---|---|---|
| Aspirin | 11.0 | 11.0 | 19.7 | 21.9 |
| Azithromycin | 11.6 | 13.0 | 12.3 | 29.2 |
| Buspirone | 17.6 | 18.6 | 18.5 | 17.9 |
| Idarubicin | 13.9 | 13.3 | 12.2 | 15.6 |
| Levofloxacin | 14.0 | 12.1 | 13.7 | 14.5 |
| Moxifloxacin | 9.7 | 9.8 | 11.3 | 14.0 |
| Pioglitazone | 44.1 | 45.6 | 44.2 | 43.6 |
| Promethazine | 17.0 | 19.4 | 17.2 | 22.1 |
| Rofecoxib | 12.2 | 12.2 | 12.9 | 15.9 |
| Sertraline | 14.0 | 16.4 | 14.5 | 16.6 |
| Bromfenac | 13.8 | 36.0 | 17.7 | 42.5 |
| Chlorpromazine | 111.1 | 97.9 | 93.2 | 96.3 |
| Clavulanate | 17.4 | 21.2 | 17.3 | 24.1 |
| Diclofenac | 15.6 | 39.0 | 16.2 | 51.2 |
| Doxorubicin | 112.8 | 94.8 | 127.8 | 101.1 |
| Flucloxacillin | 17.9 | 17.5 | 19.8 | 16.9 |
| Flutamide | 24.6 | 25.0 | 22.1 | 21.0 |
| Ibuprofen | 101.2 | 105.2 | 104.1 | 105.1 |
| Isoniazid | 57.7 | 94.1 | 48.9 | 65.3 |
| Naproxen | 101.7 | 96.2 | 99.3 | 96.1 |
| Nimesulide | 14.6 | 106.5 | 13.2 | 102.6 |
| Telithromycin | 117.1 | 99.2 | 111.1 | 115.9 |
| Trovafloxacin | 18.7 | 45.6 | 18.4 | 68.7 |
| Valproic Acid | 44.5 | 84.1 | 75.4 | 89.7 |

TABLE 7
Slope values from concentration-response data. These values were
determined by the four-parameter logistic equation as described in
Methods. Due to the method of parameterization, a negative slope
means increasing function. For treatments that did not result in a
statistically significant increase in percent LDH release from min,
slope = 0.

| Drug | Slops | Slope TNF | Slope IFN | Slope TNF/IFN |
|---|---|---|---|---|
| Aspirin | 0 | 0 | 3.013419 | 0 |
| Azithromycin | 0 | 0 | 0 | −1.8 |
| Buspirone | 0 | 0 | 0 | 0 |
| Idarubicin | 0 | 0 | 0 | 0 |
| Levofloxacin | 0 | 0 | 0 | 0 |
| Moxifloxacin | 0 | 0 | 0 | 0 |
| Pioglitazone | −16.0 | −11.9 | −14.0 | −30.1 |
| Promethazine | 0 | 0 | 0 | 0 |
| Rofecoxib | 0 | 0 | 0 | 0 |
| Sertraline | 0 | 0 | 0 | 0 |
| Bromfenac | 0 | −3.4 | −19.7 | −4.0 |
| Chlorpromazine | −10.1 | −7.6 | −12.6 | −5.3 |
| Clavulanate | 0 | −8.2 | 0 | −16.1 |
| Diclofenac | 0 | −12.5 | 0 | −11.2 |
| Doxorubicin | −2.0 | −1.5 | −1.4 | −0.9 |
| Flucloxacillin | 0 | 0 | −5.0 | 0 |
| Flutamide | −0.2 | −0.4 | −0.5 | −3.3 |
| Ibuprofen | −6.1 | −2.2 | −5.5 | −2.2 |
| Isoniazid | −32.3 | −3.1 | −43.4 | −4.7 |
| Naproxen | −7.3 | −6.6 | −8.3 | −4.0 |
| Nimesulide | 0 | −14.9 | 0 | −15.2 |
| Telithromycin | −4.2 | −3.1 | −4.0 | −2.3 |
| Trovafloxacin | −1.9 | −1.8 | −1.4 | −0.8 |
| Valproic Acid | −0.4 | −1.8 | −0.8 | −1.7 |

TABLE 8
EC50 values from concentration-response data. EC50 values were
determined from the four-parameter logistic equation as described
in Methods. NA, treatments did not result in a statistically
significant increase in percent LDH release from min. For
construction of classification models, all NA values were
considered to be zero.

| Drug | EC50 | EC50 TNF | EC50 IFN | EC50 TNF/IFN |
|---|---|---|---|---|
| Aspirin | NA | NA | 7.9 | NA |
| Azithromycin | NA | NA | NA | 207.1 |
| Buspirone | NA | NA | NA | NA |
| Idarubicin | NA | NA | NA | NA |
| Levofloxacin | NA | NA | NA | NA |
| Moxifloxacin | NA | NA | NA | NA |
| Pioglitazone | 81.9 | 87.5 | 86.8 | 88.0 |
| Promethazine | NA | NA | NA | NA |
| Rofecoxib | NA | NA | NA | NA |
| Sertraline | NA | NA | NA | NA |
| Bramfenac | NA | 43.5 | 41.7 | 32.5 |
| Chlorpromazine | 44.5 | 40.6 | 40.5 | 36.6 |
| Clavulanate | NA | 89.9 | NA | 78.0 |
| Diclofenac | NA | 28.5 | NA | 28.3 |
| Doxorubicin | 35.4 | 7.2 | 52.2 | 7.2 |
| Flucloxacillin | NA | NA | 32.0 | NA |
| Flutamide | 27.7 | 76.7 | 33.5 | 86.6 |
| Ibuprofen | 72.8 | 38.6 | 75.7 | 39.9 |
| Isoniazid | 457.1 | 507.2 | 442.4 | 272.2 |
| Naproxen | 80.8 | 32.9 | 84.4 | 29.7 |
| Nimesulide | NA | 36.9 | NA | 38.3 |
| Telithromycin | 129.7 | 95.8 | 140.5 | 101.7 |
| Trovafloxacin | 13.9 | 8.0 | 9.3 | 8.5 |
| Valproic Acid | 47.6 | 60.7 | 310.5 | 53.1 |

TABLE 9
EC10 values: the [drug]/Cmax value corresponding to 10% of
Delta (max-min). These values were determined from the equation
described in Methods. NA, treatments did not result in a
statistically significant increase in percent LDH release from
min. For construction of classification models, all NA values were
considered to be zero.

| Drug | EC10 | EC10 TNF | EC10 IFN | EC10 TNF/IFN |
|---|---|---|---|---|
| Aspirin | NA | NA | NA | NA |
| Azithromycin | NA | NA | NA | 59.2 |
| Buspirone | NA | NA | NA | NA |
| Idarubicin | NA | NA | NA | NA |
| Levofloxacin | NA | NA | NA | NA |
| Moxifloxacin | NA | NA | NA | NA |
| Pioglitazone | 71.4 | 72.8 | 74.2 | 81.8 |
| Promethazine | NA | NA | NA | NA |
| Rofecoxib | NA | NA | NA | NA |
| Sertraline | NA | NA | NA | NA |
| Bromfenac | NA | 22.6 | NA | 18.9 |
| Chlorpromazine | 35.8 | 30.5 | 34.1 | 24.1 |
| Clavulanate | NA | NA | NA | NA |
| Diclofenac | NA | 23.9 | NA | 23.2 |
| Doxorubicin | 12.0 | 1.7 | 10.6 | 0.6 |
| Flucloxacillin | NA | NA | NA | NA |
| Flutamide | NA | NA | NA | NA |
| Ibuprofen | 50.9 | 14.1 | 50.7 | 14.6 |
| Isoniazid | 427.0 | 248.0 | 420.5 | 169.7 |
| Naproxen | 59.8 | 23.6 | 64.8 | 17.3 |
| Nimesulide | NA | 31.8 | NA | 33.2 |
| Telithromycin | 77.0 | 47.7 | 80.7 | 39.0 |
| Trovafloxacin | NA | 2.3 | NA | 0.5 |
| Valproic Acid | 0.5 | 17.3 | 23.4 | 14.1 |

TABLE 10
D10 values for each drug/cytokine treatment combination.
D10 = 0 when Delta ≤ 10 LDH percentage points and
D10 = 1 when the Delta > 10 LDH percentage points.

| Drug | D10 | D10 TNF | D10 IFN | D10 TNF/IFN |
|---|---|---|---|---|
| Aspirin | 0 | 0 | 0 | 0 |
| Azithromycin | 0 | 0 | 0 | 1 |
| Buspirone | 0 | 0 | 0 | 0 |
| Idarubicin | 0 | 0 | 0 | 0 |
| Levofloxacin | 0 | 0 | 0 | 0 |
| Moxifloxacin | 0 | 0 | 0 | 0 |
| Pioglitazone | 1 | 1 | 1 | 1 |
| Promethazine | 0 | 0 | 0 | 0 |
| Rofecoxib | 0 | 0 | 0 | 0 |
| Sertraline | 0 | 0 | 0 | 0 |
| Bromfenac | 0 | 1 | 0 | 1 |
| Chlorpromazine | 1 | 1 | 1 | 1 |
| Clavulanate | 0 | 0 | 0 | 0 |
| Diclofenac | 0 | 1 | 0 | 1 |
| Doxorubicin | 1 | 1 | 1 | 1 |
| Flucloxacillin | 0 | 0 | 0 | 0 |
| Flutamide | 0 | 0 | 0 | 0 |
| Ibuprofen | 1 | 1 | 1 | 1 |
| Isoniazid | 1 | 1 | 1 | 1 |
| Naproxen | 1 | 1 | 1 | 1 |
| Nimesulide | 0 | 1 | 0 | 1 |
| Telithromycin | 1 | 1 | 1 | 1 |
| Trovafloxacin | 0 | 1 | 0 | 1 |
| Valproic Acid | 1 | 1 | 1 | 1 |

TABLE 11
R10 values are the [drug]/Cmax at which a 10 percent increase
in the LDH response above min occurs. These values were
computed using the equation listed in the methods. R10 was
considered to be 0 when D10 = 0.

| Drug | R10 | R10 TNF | R10 IFN | R10 TNF/IFN |
|---|---|---|---|---|
| Aspirin | 0 | 0 | 0 | 0 |
| Azithromycin | 0 | 0 | 0 | 333.9 |
| Buspirone | 0 | 0 | 0 | 0 |
| Idarubicin | 0 | 0 | 0 | 0 |
| Levofloxacin | 0 | 0 | 0 | 0 |
| Moxifloxacin | 0 | 0 | 0 | 0 |
| Pioglitazone | 80.1 | 84.5 | 84.6 | 87.3 |
| Promethazine | 0 | 0 | 0 | 0 |
| Rofecoxib | 0 | 0 | 0 | 0 |
| Sertraline | 0 | 0 | 0 | 0 |
| Bromfenac | 0 | 40.6 | 0 | 28.9 |
| Chlorpromazine | 36.0 | 31.5 | 34.8 | 25.3 |
| Clavulanate | 0 | 0 | 0 | 0 |
| Diclofenac | 0 | 28.3 | 0 | 26.7 |
| Doxorubicin | 12.4 | 2.0 | 9.7 | 0.7 |
| Flucloxacillin | 0 | 0 | 0 | 0 |
| Flutamide | 0 | 0 | 0 | 0 |
| Ibuprofen | 51.7 | 14.4 | 51.3 | 15.5 |
| Isoniazid | 440.9 | 268.1 | 434.0 | 208.7 |
| Naproxen | 60.5 | 24.3 | 65.9 | 18.4 |
| Nimesulide | 0 | 32.0 | 0 | 33.5 |
| Telithromycin | 76.6 | 50.5 | 81.3 | 39.3 |
| Trovafloxacin | 0 | 4.8 | 0 | 1.2 |
| Valproic Acid | 16.6 | 23.3 | 51.3 | 18.6 |

TABLE 12
Q, EC50 quotient, EC10 quotient, R10 quotient and Deltadiff
values for each drug. These values were determined as
described in Methods. The EC50, EC10 and R10 quotient
covariates shown in the table represent the ratio of the
drug/TNF concentration-response curve to the drug/VEH curve,
and Deltadiff represents the difference between the Deltas of
drug/TNF concentration-response curve and the drug/VEH curve

| | | EC50 | EC10 | R10 | |
|---|---|---|---|---|---|
| Drug | Q | Quotient | quotient | quotient | Deltadiff |
| Aspirin | 0 | 0 | 0 | 0 | 0 |
| Azithromycin | 0 | 0 | 0 | 0 | 0 |
| Buspirone | 0 | 0 | 0 | 0 | 0 |
| Idarubicin | 0 | 0 | 0 | 0 | 0 |
| Levofloxacin | 1 | 0 | 0 | 0 | 0 |
| Moxifloxacin | 0 | 0 | 0 | 0 | 0 |
| Pioglitazone | 1 | 0.9 | 1.0 | 0.9 | 0.8 |
| Promethazine | 0 | 0 | 0 | 0 | 0 |
| Rofecoxib | 0 | 0 | 0 | 0 | 0 |
| Sertraline | 1 | 0 | 0 | 0 | 0 |
| Bromfenac | 0 | 0 | 0 | 0 | 22.6 |
| Chlorpromazine | 1 | 1.1 | 1.2 | 1.1 | −15.3 |
| Clavulanate | 0 | 0 | 0 | 0 | 3.1 |
| Diclofenac | 0 | 0 | 0 | 0 | 21.0 |
| Doxorubicin | 0 | 4.9 | 7.0 | 6.1 | −15.2 |
| Flucloxacillin | 1 | 0 | 0 | 0 | 0 |
| Flutamide | 0 | 0 | 0 | 0 | 0.4 |
| Ibuprofen | 1 | 1.9 | 3.6 | 3.6 | 3.9 |
| Isoniazid | 0 | 0.9 | 1.7 | 1.6 | 38.8 |
| Naproxen | 0 | 2.5 | 2.5 | 2.5 | −9.3 |
| Nimesulide | 0 | 0 | 0 | 0 | 92.3 |
| Telithromycin | 1 | 1.4 | 1.6 | 1.5 | −17.1 |
| Trovafloxacin | 0 | 0 | 0 | 0 | 27.4 |
| Valproic Acid | 1 | 0.8 | 0.03 | 0.7 | 37.1 |

TABLE 13
The values for the categorical variable TNF change for each drug.
TNF change = 1 when the drug/VEH curve is flat (no LDH
response) and the drug/TNF curve is sigmoidal. TNF change =
0 in all other situations. See Methods for definition.

| Drug | TNF change |
|---|---|
| Aspirin | 0 |
| Azithromycin | 0 |
| Buspirone | 0 |
| Idarubicin | 0 |
| Levofloxacin | 0 |
| Moxifloxacin | 0 |
| Pioglitazone | 0 |
| Promethazine | 0 |
| Rofecoxib | 0 |
| Sertraline | 0 |
| Bromfenac | 1 |
| Clavulanate | 0 |
| Diclofenac | 1 |
| Doxorubicin | 0 |
| Flucloxacillin | 0 |
| Flutamide | 0 |
| Ibuprofen | 0 |
| Isoniazid | 0 |
| Naproxen | 0 |
| Nimesulide | 1 |
| Telithromycin | 0 |
| Trovafloxacin | 1 |
| Valproic Acid | 0 |

Delta was defined as max-min. In addition to the covariates determined from concentration-response curves, Cmax was considered as another base covariate.

Similar to EC50, the covariate EC10 represents the [drug]/Cmax value associated with a 10% increase above min relative to max and was determined by the equation:

EC10=D10·EC50·9^{1/slope}

where D10 is a categorical variable related to reaching a threshold LDH response above which a drug is classified as positively associated with IDILI. D10 is defined as 0 if Delta<10% LDH release and as 1 if Delta≥10% LDH release.

The base covariate R10 represents the [drug]/Cmax value associated with an increase in 10 LDH percentage points above min for a particular treatment condition and was determined by the equation:

$$R 10 = EC 50 \cdot {[ \frac{Delta}{10} - 1 ]}^{1 / slope}$$

R10 was considered to be 0 when the Delta<10% LDH (i.e. when D10=0).

From the base covariates defined above, several other covariates were derived. These included EC50 quotient, EC10 quotient, R10 quotient, Deltadiff and TNF change. Each of these “derived” covariates is explained in more detail below.

EC50 quotient, EC10 quotient and R10 quotient represent the ratio between the EC50, EC10 or R10 of the drug/cytokine concentration-response curve and the respective value for the drug/VEH concentration-response curve. In some instances, the value derived from this calculation is indeterminate (i.e. when the denominator=0). In order to incorporate the quotient values into the classification models described below, the categorical variable “Q” was used to eliminate the possibility of the quotient being indeterminate. Q is defined as 0 if Delta VEH and/or Delta cytokine is <10% LDH and 1 if both Delta VEH and Delta cytokine are ≥10% LDH. For the purpose of calculating EC50 quotient the following condition was applied: if Q=0, then EC50 quotient=0 and if Q=1, then EC50 quotient=EC50 VEH/EC50 cytokine. The same condition was applied for calculation of EC10 quotient and R10 quotient.

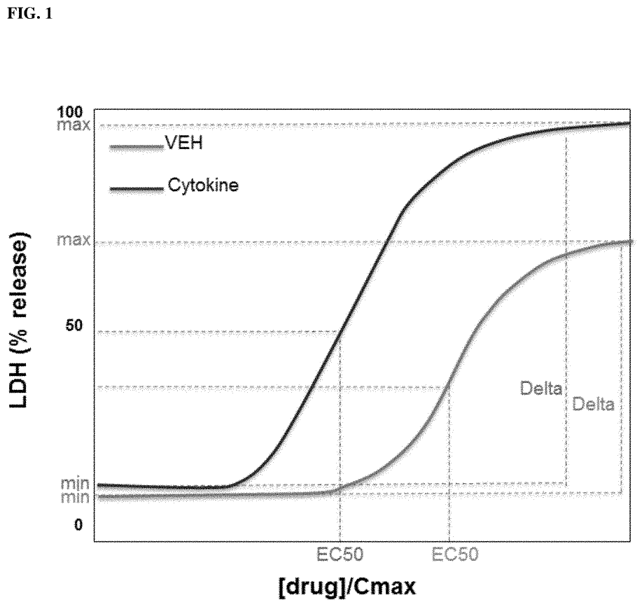

Deltadiff represents the difference between the Delta of the drug/cytokine concentration-response curve and the Delta of the drug/VEH curve. In other words, Deltadiff=(Delta cytokine)−(Delta VEH). FIG. 1 graphically illustrates several of the covariates defined above. Tables 5-13 list the values of all of the covariates computed in this study. In addition to the covariates derived from the concentration-response curves, the maximal therapeutic plasma drug concentration (Cmax) in human patients was used in some models (Table 1).

TNF change is a categorical variable related to the alteration in the drug-induced cytotoxic response in the presence and absence of TNF, determined as:

TNF change=D10TNF−D10VEH

Recall that D10 is defined as 0 if Delta 10% change in LDH release, and D10 is defined as 1 if Delta>10% change in LDH release. Accordingly, TNF change=1 if the TNF curve has a Delta>10% LDH release and the VEH curve has a Delta≤10% LDH release; TNF change=0 in all other situations.

Classification modeling using defined covariates. In the second phase of analysis, classification models were developed using logistic regression with covariates as independent variables in the analysis and known IDILI classification as the dependent variable. Ability to classify drugs accurately was evaluated using the known IDILI classifications shown in Table 1. A model selection process was used to determine if a covariate or set of covariates is associated with IDILI liability. Covariates were first evaluated individually to determine how well a particular covariate classified drugs according to IDILI liability, and then covariates were evaluated in combination. Combinations of covariates were selected to maximize the ability of the model to distinguish between drugs associated or not with IDILI. Specifically, covariates that describe changes in efficacy (Delta, Deltadiff, etc.) were paired with covariates that describe changes in potency (EC50, EC10, R10, etc.) to find covariate combinations that led to models that most accurately discriminated between drugs that are and are not associated with IDILI. The best-fit logistic regression models were used as classification models to compute a probability that a given drug is associated with IDILI. The logistic regression models follow the equation:

$${\hat{y}}_{i} - prob ( IDILI - 1 \vert {x}_{i} ) - \frac{{e}^{{β}_{0} \to \sum {β}_{i} {x}_{i}}}{1 + {e}^{{β}_{0} ← \sum {β}_{i} {x}_{i}}}$$

where ŷᵢ is the calculated (predicted) probability that drug i with a vector of covariates xᵢ is associated with IDILI. The β coefficients (β₀, the regression intercept; and βᵢ, the regression slopes for model covariates, xᵢ) were derived from the logistic regression models using 1) combinations of the covariates (xᵢ) generated by concentration-response modeling for each of the 24 drugs evaluated in this study, and 2) the true IDILI classification for the dependent variable yᵢ (i.e., 1 for IDILI+drugs and 0 for IDILI-drugs). For treatments that did not result in a significant change in LDH above min (p>0.01), a value of 0 was assigned for covariates derived from EC50 for purposes of calculating βᵢ. The regression coefficients (βᵢ) were calculated using Firth's method, which eliminates bias when estimating the value βᵢ (Firth 1993). Firth's method was necessary since many of the covariates used in this study exhibited quasi-complete separation. This occurs when a covariate almost perfectly separates observations into the appropriate categories. In this study, several covariates almost completely separated drugs according to their IDILI liability. When separation or quasi-complete separation occurs, use of the standard method (i.e., maximum likelihood estimation) provides biased, unreliable estimates of βᵢ. Firth's method uses a penalized likelihood regression to rectify this and is an appropriate method to use for estimating βᵢ when quasi-complete separation of data occurs (Firth, 1993). All logistic regression models were computed using R statistical software (R package “logistf”) (R Core Team, 2015, Heinze et al. 2013).

Receiver operating characteristic (ROC) analysis. In the third phase of the statistical approach, the classification models, generated as logistic regression models using single covariates or combinations of covariates, were evaluated by ROC analysis to determine which model and corresponding sets of covariates led to the most accurate classification of drugs according to their potential to cause IDILI. An ROC curve was created for each model by graphing the true positive rate (sensitivity; i.e., proportion of drugs correctly classified as associated with IDILI) against the false positive rate (1-specificity; i.e., proportion of drugs incorrectly classified as associated with IDILI) at various probability cutoff thresholds (k). ROC curves were generated using the R package, pROC (R Core Team, 2015, Robin et al. 2011). An area under the curve (AUC) and confidence interval was computed for each ROC curve (where each logistic regression model has one ROC curve). Plots depicting the AUCs and 95% confidence intervals of the ROC curves were generated for the purpose of comparing multiple logistic regression models using the R package, Metafor (Viechtbauer et al. 2010). Corresponding to each ROC curve is an optimal threshold value (k*), the threshold yielding the highest point of accuracy on the curve (i.e., the point nearest the point (1,1) on the curve). Thus, each ROC curve has a corresponding AUC and an optimal cutoff (k*) that corresponds to the highest point of accuracy on that ROC curve.

Combinations of covariates were strategically selected for evaluation based on what was deemed to lead to the most accurate classification of drugs. ROC curves and corresponding whisker plots were generated to illustrate graphically the ability of each classification model to classify drugs accurately. This allowed for selection of optimal set(s) of covariates for accurate drug classification according to IDILI liability. Our goal was to achieve a classification model and a corresponding set of covariates with an AUC as close to 1 as possible with the narrowest 95% confidence interval. A model that is able to classify drugs perfectly according to their potential to cause IDILI would have an ROC curve with an AUC=1. DeLong's method was used to determine if there were statistically significant differences among ROC curves (DeLong et al. 1988).

A separate classification analysis was also performed to evaluate the ability of Cmax to classify a larger set of drugs with known IDILI potential. Cmax values were obtained for 272 drugs from a study conducted by Xu et al. (2008) and evaluated using ROC analysis.

### Example 2: Results

### Drug/Cytokine Cytotoxicity: Concentration-Response In Vitro

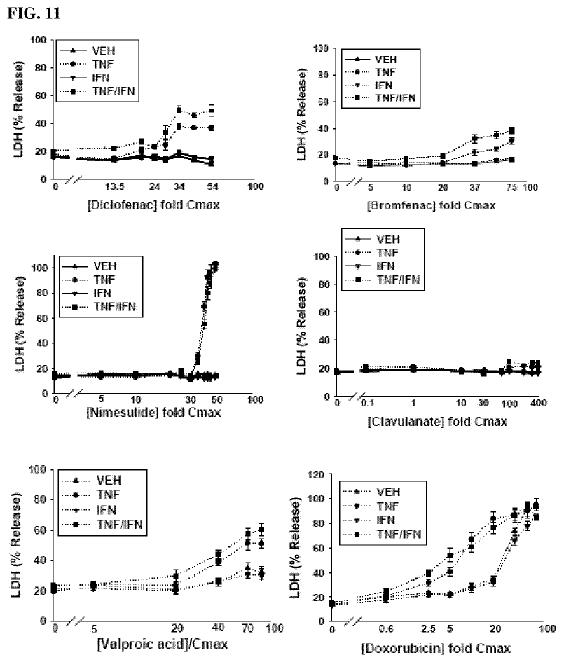

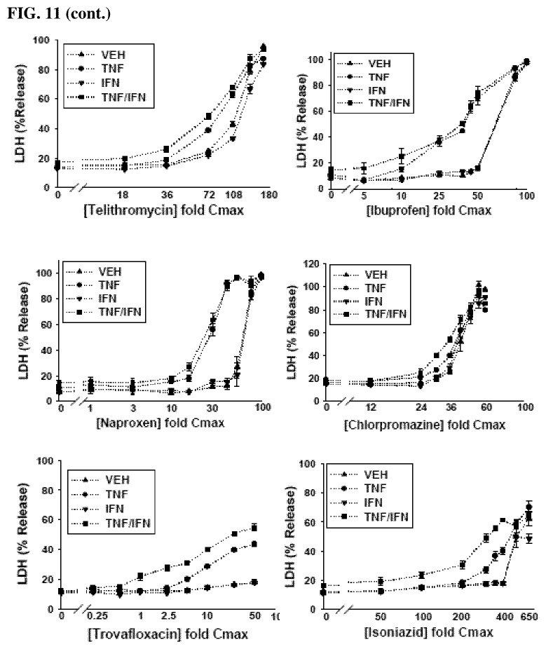

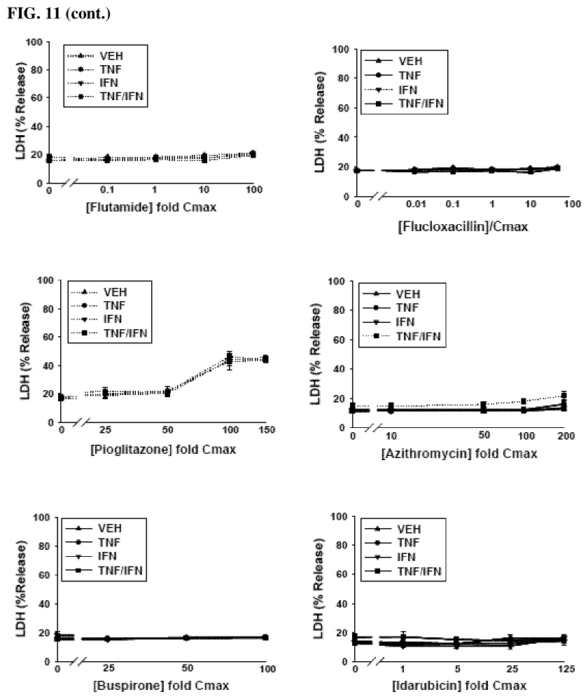

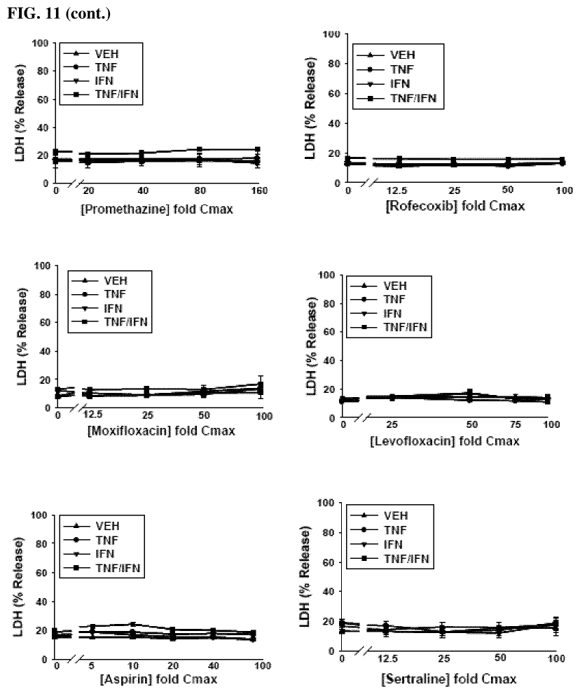

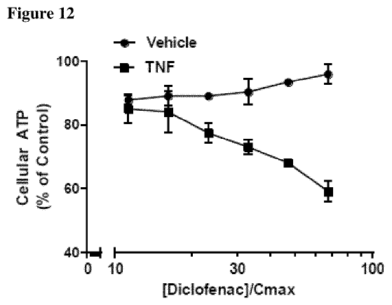

HepG2 cells were treated with various concentrations of a drug alone or in combination with TNF and/or IFN, and cytotoxicity was assessed 24 hours later as increased LDH activity in the culture medium. Detailed cytotoxicity concentration-response curves were generated for 24 drugs (Table 1): 14 drugs that are associated with IDILI and 10 that are not (negative comparators) (FIG. 2, FIG. 11). Of the 14 drugs associated with IDILI, almost all synergized with cytokines in causing cell death (FIG. 2). Four of these (diclofenac, bromfenac, nimesulide and clavulanate) caused no cytotoxicity on their own but synergized with TNF to cause cytotoxicity. Nine IDILI-associated drugs led to a statistically significant increase in LDH release (relative to no drug) in the absence of cytokines (valproic acid, doxorubicin, telithromycin, ibuprofen, naproxen, chlorpromazine, flutamide, trovafloxacin and isoniazid) Interestingly TNF significantly enhanced the cytotoxicity mediated by eight of these drugs (valproic acid, doxorubicin, telithromycin, ibuprofen, naproxen, chlorpromazine, trovafloxacin and isoniazid). In contrast to TNF, IFN did not interact with any drug to cause cytotoxicity. However, coexposure to IFN enhanced the cytotoxic interaction between TNF and several of the drugs (diclofenac, bromfenac, trovafloxacin, valproic acid, chlorpromazine, telithromycin and isoniazid). Two of the 14 IDILI-associated drugs (flutamide and flucloxacillin) did not synergize with cytokines to kill HepG2 cells. Of the 10 negative comparators, pioglitazone was the only drug that caused cytotoxicity on its own; however, this effect was not enhanced by the addition of cytokines. With the exception of azithromycin, which was modestly cytotoxic in the presence of TNF/IFN, none of the remaining negative comparator drugs synergized with cytokines to kill HepG2 cells (FIG. 2).

### Classification Models and ROC Analysis

Data used in the classification models consisted of covariate data generated as described above (i.e., parameters of the concentration-response curves for each of the 24 drugs and additional variables derived from these parameters) in addition to daily dose and Cmax for each drug. Daily dose and Cmax values are shown in Table 1. Tables 5-13 summarize parameters of the concentration-response curves for each drug and all derived covariates. The model selection process, described below, involved estimation of numerous logistic regression models beginning with individual models for each covariate alone and then with models populated with combinations of covariates Table 14. All classification models were compared for their ability to classify the 24 drugs using ROC analysis. Findings are described below.

Cmax is Moderately Associated with IDILI Potential

IDILI reactions were once thought not to be dose-related; however, the observation that most drugs that have been withdrawn from the market or have received a black box warning due to IDILI were prescribed at doses greater than 50 mg/day suggested that daily dose plays some role in the propensity of a drug to cause IDILI (Uetrecht 1999). Based on this observation, we evaluated how accurately the daily dose or the Cmax of a drug classifies drugs listed in Table 1 according to their potential to cause IDILI. Logistic regression modeling and ROC analysis as described in Methods were used. The AUC of the ROC curve generated for the model incorporating daily dose is 0.64 with a 95% confidence interval of [0.37, 0.9] (FIG. 3). Since the 95% confidence interval for the ROC curve from the daily dose model contains the value 0.5, representing no better than random classification, it cannot be concluded that the magnitude of the daily dose is predictive of IDILI for this drug set. A larger set of drugs may be needed to determine if daily dose can predict IDILI.

The AUC of the ROC curve generated for the model incorporating Cmax for our set of 24 drugs is 0.80, with a 95% confidence interval of [0.61, 0.98] (FIG. 3). Similar results were obtained by Shah et al (2015) for a set of 125 drugs. These results suggest that Cmax is associated with IDILI.

To determine if our set of 24 drugs is representative of a larger set of drugs and to evaluate further the ability of Cmax to predict IDILI liability, Cmax values were obtained for 272 drugs from a study conducted by Xu et al. (2008) and converted to μM units. Cmax is a significant predictor of IDILI for this larger dataset (β_{Cmax}=0.044, p<0.001). The AUC of the ROC curve generated from this larger set of drugs is 0.70 with a confidence interval of [0.64, 0.76]. The ROC curves derived from the set of 24 drugs and from the set of 272 drugs are depicted along with their 95% confidence intervals in FIGS. 9A and 9B. The confidence interval corresponding to the ROC curve derived from the set of 272 drugs (shaded red) is contained within the confidence interval for the ROC curve derived from the set of 24 drugs (shaded grey). This suggests that the smaller set of drugs adequately represents the relationship between Cmax and IDILI potential seen in a much larger set of drugs.

### ROC Analysis of Models Incorporating the Base Covariates

Almost all of the 14 IDILI-associated drugs synergized with TNF to cause death of HepG2 cells, and some of them were cytotoxic by themselves (FIG. 2). These results suggested that cytotoxic synergy with TNF might be associated with IDILI liability. Accordingly, classification models were constructed using base covariates from the concentration-response curves to determine whether the presence of TNF improved a model's ability to classify drugs according to IDILI liability. The base covariates were modeled for each drug individually; base covariates that were at least moderately associated with IDILI liability included Delta VEH, Delta TNF, EC50 VEH, EC50 TNF, EC10 VEH, EC10 TNF, R10 VEH and R10 TNF. AUCs and 95% confidence intervals are shown in FIG. 4A for each of these covariates. It can be seen that the confidence interval for each of these covariates does not contain the value 0.5, indicating a significantly better than random ability to classify the 24 drugs according to IDILI potential. The model incorporating Delta TNF produced the ROC curve with the greatest AUC (0.93) and narrowest 95% confidence interval (0.83, 1.00) suggesting that, of these models, it provided the most accurate classification of the drugs (FIG. 4A). Furthermore, the base covariates that described the response to drug/TNF (labeled ‘TNF’) led to models that produced ROC curves that had improved AUCs with narrower confidence intervals than those that described the response to drug alone (i.e., labeled VEH) (FIG. 4A, B).

### ROC Analysis of Models Incorporating Derived Covariates

Probability models were also generated using the individual covariates that were derived from the base covariates: EC50 quotient, EC10 quotient, R10 quotient, and Deltadiff. In FIGS. 5A and B, the EC50, EC10 and R10 quotient covariates represent the ratio of the drug/TNF concentration-response curve to the drug/VEH curve, and Deltadiff represents the difference between the Deltas from the drug/TNF concentration-response curve and the drug/VEH curve. Each of these covariates (except Deltadiff) was moderately associated with IDILI liability (FIG. 5A, B); however, the ROC curves generated based on these models did not have greater AUCs or narrower confidence intervals than the models produced by incorporating the initial base covariates (compare FIGS. 4 and 5).

### Addition of IFN Data Did not Improve the Classification of Drugs

None of the drugs synergized with IFN in the absence of TNF to cause cytotoxicity, but several IDILI-associated drugs synergized with IFN in the presence of TNF (FIG. 2). Accordingly, we examined whether incorporation of the TNF/IFN responses would improve the performance of models that employed only TNF responses. The drug/TNF/IFN models tended to have smaller AUCs and larger confidence intervals than the drug/TNF models (FIGS. 10A and 10B), indicating that the addition of data describing the IFN response did not enhance the ability of models to classify drugs. While under these parameters, IFN data did not improve the classification of drugs, future refining of the assay may render IFN useful.

### ROC Analysis of Models Incorporating Combinations of the Base and Derived Covariates

Although it was illustrative to evaluate the base and derived covariates individually, we hypothesized that incorporation of several covariates into a model would lead to more accurate classification of drugs than incorporation of a single covariate. Accordingly, various combinations of the base and derived covariates were evaluated to identify a set of covariates that led to the most accurate drug classification. Combining base and derived covariates led to several models with greater AUCs and narrower confidence intervals than the models incorporating only a single covariate. A representative set including the best performing models is presented in FIG. 6. Furthermore, when Cmax was added as a covariate, it improved the performance (AUC and confidence interval) of some models but not others (FIG. 6). Table 14 shows the coefficients (beta values) and their p-values for the models shown in FIG. 6.

Some of the combination models were associated with remarkably high AUCs, and some of these were associated with small confidence intervals. There were no statistically significant differences among the models with an AUC>0.95 as determined by DeLong's method for comparing ROC curves (p>0.05). The ROC curves that met this criterion (AUC>0.95) are shown in FIG. 7.

### REFERENCES

1. Aithal G P, Watkins P B, Andrade R J, Larrey D, Molokhia M, Takikawa H, Hunt C M, Wilke R A, Avigan M, Kaplowicz N, et al. (2011) Case definition and phenotype standardization in drug-induced liver injury. Clin Pharmacol Ther 89(6):806-15.
2. Aleo M D, Luo Y, Swiss R, Bonin P D, Potter D M, and Will Y (2014) Human druginduced liver injury severity is highly associated with dual inhibition of liver mitochondrial function and bile salt export pump. Hepatology 60:1015-1022.
3. Ball A L, Kamalian L, Alfirevic A, Lyon J J, and Chadwick A E (2016) Identification of the additional mitochondrial liabilities of 2-hydroxyflutamide when compared with its parent compound, flutamide in HepG2 cells. Toxicol Sci 153:341-351.
4. Barpe, D., Rosa, D., Froehlich, P. 2010. Pharmacokinetic evaluation of doxorubicin plasma levels in normal and overweight patients with breast cancer and simulation of dose adjustment by different indexes of body mass. Eur. J. Pharm. Sci. 41, 458-463.
5. Beggs, K., Fullerton, A., Miyakawa, K., Ganey, P. and Roth, R. 2014. Molecular mechanisms of hepatocellular apoptosis induced by trovafloxacin-tumor necrosis factor-alpha interaction. Toxicol. Sci. 137, 91-101.
6. Beggs, K., Maiuri, A., Fullerton, A., Poulsen, K., Breier, A., Ganey, P., Roth, R. 2015. Trovafloxacin-induced replication stress sensitizes HepG2 cells to tumor necrosis factor-alpha-induced cytotoxicity mediated by extracellular signal-regulated kinase and ataxia telangiectasia and Rad3-related. Toxicology. 331, 35-46.
7. Bengtsson, F., Floren, C., Hagerstrand, I., Soderstrom, C., Aberg, T. 1985. Flucloxacillin-induced cholestatic liver damage. Scand. J. Infect. Dis. 17, 125-128.
8. Beraldo, D., Melo, J., Bonfim, A., Teixeira, A., Teixeira, R., Duarte, A. Acute cholestatic hepatitis caused by amoxicillin/clavulanate. 2013 World J Gastroenterol. 19:8789-92.
9. Bramlage, P., Goldis, A. 2008. Bioequivalence study of three ibuprofen formulations after single dose administration in healthy volunteers. BMC Pharmacol. 8, 1-9.
10. Brandon, R., Eadie, M., Curran, A., Nolan, P., Presneill, J. 1986. A new formulation of aspirin: bioavailability and analgesic efficacy in migraine attacks. Cephalalgia 6, 19-27.
11. Brogden R N and Clissold S P (1989) Flutamide. A preliminary review of its pharmacodynamics and pharmacokinetic properties, and therapeutic efficacy in advanced prostatic cancer. Drugs 38:185-203.
12. Buchweitz J P, Ganey P E, Bursian S J, and Roth R A (2002) Underlying endotoxemia augments toxic responses to chlorpromazine: is there a relationship to drug idiosyncrasy? J Pharmacol Exp Ther 300:460-467.
13. Chakraborty, M., Fullerton, A., Semple, K., Chea, L., Proctor, W., Bourdi, M., Kleiner, D., Zeng, X., Ryan, P., Dagur, P., Berkson, J., Reilly, T., Pohl, L. 2015. Drug-induced allergic hepatitis developed in mice when myeloid-derived suppressor cells were depleted prior to halothane treatment. Hepatology. 62: 546-557.
14. Chen M, Borlak J, and Tong W (2013) High lipophilicity and high daily dose of oral medications are associated with significant risk for drug-induced liver injury. Hepatology 58:388-396.
15. Chen M, Tung C W, Shi Q, Guo L, Shi L, Fang H, Borlak J, and Tong W (2014) A testing strategy to predict risk for drug-induced liver injury in humans using highcontent screen assays and the ‘rule-of-two’ model. Arch Toxicol 88:1439-1449.
16. Choi J M, Oh S J, Lee J Y, Jeon J S, Ryu C S, Kim Y M, Lee K, and Kim S K (2015) Prediction of drug-induced liver injury in HepG2 cells cultured with human liver microsomes. Chem Res Toxicol 28:872-885.
17. Chu X, Korzekwa K, Elsby R, Fenner K, Galetin A, Lai Y, Matsson P, Moss A, Nagar S, Rosania G R, et al.; International Transporter Consortium (2013) Intracellular drug concentrations and transporters: measurement, modeling, and implications for the liver. Clin Pharmacol Ther 94:126-141.
18. Church, R. J., Gatti, D. M, Urban, T., Long, N., Yang, X., Shi, Q., Eaddy, J., Mosedale, M., Ballard, S., Churchill, G., Navarro, V., Watkins, P., Threadgill, D., Harrill, A. 2015. Sensitivity to hepatotoxicity due to epigallocatechin gallate is affected by genetic background in diversity outbred mice. Food Chem Toxicol. 76:19-26.
19. Cosgrove, B., King, B., Hasan, M., Alexopoulos, L., Farazi, P., Hendriks, B., Griffith, L., Sorger, P., Tidor, B., Xu, J., Lauffenburger, D. 2009. Synergistic drug-cytokine induction of hepatocellular death as an in vitro approach for the study of inflammation-associated idiosyncratic drug hepatotoxicity. Toxicol. Appl. Pharmacol. 237, 317-330.
20. DeLong, E., DeLong, D., Clarke-Pearson, D. 1988. Comparing the areas under two or more correlated receiver operating characteristic curves: a nonparametric approach. Biometrics. 44, 837-845.
21. Deng X, Stachlewitz R F, Liguori M J, Blomme E A, Waring J F, Luyendyk J P, Maddox J F, Ganey P E, and Roth R A (2006) Modest inflammation enhances diclofenac hepatotoxicity in rats: role of neutrophils and bacterial translocation. J Pharmacol Exp Ther 319:1191-1199.
22. Dugan, C., Fullerton, A., Roth, R., Ganey, P. 2011. Natural Killer Cells Mediate Severe Liver Injury in a Murine Model of Halothane Hepatitis. Toxicol. Sci. 120, 507-518.
23. Enat, R., Pollack, S., Ben-Arieh, Y., Livni, E., Barzilai, D. 1980. Cholestatic jaundice caused by cloxacillin: macrophage inhibition factor test in preventing rechallenge with hepatotoxic drugs. Br. Med. J. 280, 982-983.
24. Firth, D. 1993. Bias reduction of maximum likelihood estimates. Biometrika. 80, 27-38.
25. Fredriksson, L., Herpers, B., Benedetti, G., Matadin, Q., Puigvert, J., de Bont, H., Dragovic, S., Vermeulen, N., Commandeur, J., Danen, E., de Graauw, M. van de Water, B. 2011. Diclofenac inhibits tumor necrosis factor-alpha-induced nuclear factor-kappaB activation causing synergistic hepatocyte apoptosis. Hepatology 53, 2027-2041.
26. Fredriksson, L., Wink, S., Herpers, B., Benedetti, G., Hadi, M., de Bont, H., Groothuis, G., Luijten, M., Danen, E., de Graauw, M., Meerman, J., van de Water B. 2014. Drug-induced endoplasmic reticulum and oxidative stress responses independently sensitize toward TNFα-mediated hepatotoxicity. Toxicol. Sci. 140, 144-159.
27. Gottesdiener K., Agrawal N., Porras A., Wong P., Rogers J., Gertz B., Redfern J., Marbury T. 2003. Effects of Renal Insufficiency and Hemodialysis on the Pharmacokinetics of Rofecoxib. Am. J. Ther. 10, 252-258.
28. Gumbhir-Shah, K., Cevallos, W., DeCleene, S., Halstenson, C., Korth-Bradley, J. 1997. Absolute bioavailability of bromfenac in humans. Ann. Pharmacother. 31, 395-399.
29. Han Y, Rogers N, and Ransohoff R M (1999) Tumor necrosis factor-alpha signals to the IFN-gamma receptor complex to increase StatIalpha activation. J Interferon Cytokine Res 19:731-740.
30. Hassan, F., Morikawa, A., Islam, S., Tumurkhuu, G., Dagvadorj, J., Koide, N., Naiki. Y., Mori, I., Yoshida, T., Yokochi, T. 2008. Lipopolysaccharide augments the in vivo lethal action of doxorubicin against mice via hepatic damage. Clin Exp Immunol. 151, 334-340.
31. Heinze, G., Ploner, M., Dunkler, D., Southworth, H. 2013. Logistf: Firth's bias reduced logistic regression. R package version 1.21.
32. Hu, G., Dai, Z., Long, L., Han, Y., Hou, S., Wu, L. 2002. Bioequivalence of clavulanate potassium and amoxicillin (1:7) dispersible tablets in healthy volunteers. J. Huazhong Univ. Sci. Technolog. Med. Sci. 22, 224-227.
33. Khetani S R, Kanchagar C, Ukairo O, Krzyzewski S, Moore A, Shi J, Aoyama S, Aleo M, and Will Y (2013) Use of micropatterned cocultures to detect compounds that cause drug-induced liver injury in humans. Toxicol Sci 132:107-117.
34. Low Y, Uehara T, Minowa Y, Yamada H, Ohno Y, Urushidani T, Sedykh A, Muratov E, Kuz'min V, Fourches D, et al. (2011) Predicting drug-induced hepatotoxicity using QSAR and toxicogenomics approaches. Chem Res Toxicol 24:1251-1262.
35. Lu, J., Jones, A., Harkema, J., Roth, R., Ganey, P. Amiodarone exposure during modest inflammation induces idiosyncracy-like liver injury in rats: role of tumor necrosis factor-alpha. Toxicol. Sci. 125, 126-133.
36. Lucena, M., Molokhia, M., Shen, Y., Urban, T., Aithal, G., Andrade, R., Day, C., Ruiz-Cabello, F., Donaldson, P., Stephens, C., Pirmohamed, M., Romero-Gomez, M., Navarro, J., Fontana, R., Miller, M., Groome, M., Bondon-Guitton, E., Conforti, A., Stricker, B., Carvajal, A., Ibanez, L., Yue, Q., Eichelbaum, M., Floratos, A., Pe'er, I., Daly, M., Goldstein, D., Dillon, J., Nelson, M., Watkins, P., Daly, A. 2011. Susceptibility to amoxicillin-clavulanate-induced liver injury is influenced by multiple HLA class I and II alleles. Gastroenterology. 141, 338-347.
37. Maiuri A., Breier, A., Gora, L., Parkins, R., Ganey, P., Roth, R. 2015. Cytotoxic Synergy Between Cytokines and NSAIDs Associated with Idiosyncratic Hepatotoxicity is Driven by Mitogen-activated Protein Kinases. Toxicol. Sci. 146, 265-280.
38. Metushi I G, Hayes M A, and Uetrecht J (2015) Treatment of PD-1(2/2) mice with amodiaquine and anti-CTLA4 leads to liver injury similar to idiosyncratic liver injury in patients. Hepatology 61(4):1332-42.
39. Morgan R E, van Staden C J, Chen Y, Kalyanaraman N, Kalanzi J, Dunn R T, 2nd, Afshari C A, and Hamadeh H K (2013) A multifactorial approach to hepatobiliary transporter assessment enables improved therapeutic compound development. Toxicol Sci 136:216-241.
40. Moseley, R. Hepatotoxicity of antimicrobials and antifuncal agents. 2013. In, Kaplowitz, N., Deleve, L., eds. Drug-induced liver disease. 3^{rd} ed. Amsterdam: Elsevier, 463-482.
41. Ostapowicz G., Fontana R., Schiodt F., Larson A., Davern T., Han S., McCashland T., Shakil A., Hay J., Hynan L., Crippin J., Blei A., Samuel G., Reisch J., Lee W. 2002. Results of a prospective study of acute liver failure at 17 tertiary care centers in the United States. Ann. Intern. Med. 137, 947-954.
42. Pardoll, D. 2012. The blockade of immune checkpoints in cancer immunotherapy. Nat. Rev. Cancer. 12, 252-264.
43. Pinsky, M., Vincent, J., Alegre, M., Dupont, E. 1993. Serum cytokine levels in human septic shock. Chest 103, 565-575.
44. R Core Team. 2015. R: A language and environment for statistical computing. R Foundation for Statistical Computing, Vienne, Austria.
45. Rha, J., Jang, I., Lee, K., Chong, W., Shin, S., Lee, N., Myung, H. 1993. Pharmacokinetic comparison of two valproic acid formulations—a plain and a controlled release enteric-coated tablets. J. Korean Med. Sci. 8, 251-256.
46. Ritz, C., Streibig, J. 2005. Bioassay Analysis using R. J. Statist. Software, Vol 12, Issue 5.
47. Robin, X., Turck, N., Hainard, A., Tiberti, N., Lisacek, F., Sanchez, J., Müller. 2011. pROC: an open-source package for R and S+ to analyze and compare ROC curves. BMC Bioinformatics. 12, 77.
48. Robinson C M, Shirey K A, and Carlin J M (2003) Synergistic transcriptional activation of indoleamine dioxygenase by IFN-gamma and tumor necrosis factor-alpha. J Interferon Cytokine Res 23:413-421.
49. Roder, B., Frimodt-Moller, N., Espersen, F., Rasmussen, S. 1995. Dicloxacillin and flucloxacillin: pharmacokinetics, protein binding and serum bactericidal titers in healthy subjects after oral administration. Infection. 23, 107-112.
50. Roth, R., Ganey, P. 2011. Animal models of idiosyncratic drug-induced liver injury—current status. Crit. Rev. Toxicol. 41, 723-739.
51. Roth R A, Maiuri A R, and Ganey P E (2017) Idiosyncratic drug-induced liver injury: is drug-cytokine interaction the linchpin? J Pharmacol Exp Ther 360:461-470.
52. Sakatis M Z, Reese M J, Harrell A W, Taylor M A, Baines I A, Chen L, Bloomer J C, Yang E Y, Ellens H M, Ambroso J L, et al. (2012) Preclinical strategy to reduce clinical hepatotoxicity using in vitro bioactivation data for 0.200 compounds. Chem Res Toxicol 25:2067-2082.
53. Sánchez-Ruiz-Granados, E., Bejarano-García, A., Uceda-Torres, E. Recurrent cholestasis by amoxicillin-clavulanic acid: the importance of a correct diagnosis of hepatotoxicity. 2012 Rev Esp Enferm Dig. 104: 616-7.\
54. Schadt S, Simon S, Kustermann S, Boess F, McGinnis C, Brink A, Lieven R, Fowler S, Youdim K, Ullah M, et al. (2015) Minimizing DILI risk in drug discovery—a screening tool for drug candidates. Toxicol In Vitro 30 (Pt B):429-437.
55. Setiawati, E., Deniati, S., Yunaidi, D., Handayani, L., Harinanto, G., Santosos, I., Purnomo, Sari, A., Rimainar, A. 2009. Bioequivalence study with two naproxen sodium tablet formulations in healthy subjects. J. Bioequivalence Bioavailability 1, 28-33.
56. Shah, F., Leung, L., Barton, H. A., Will, Y., Rodrigues, A. D., Greene, N., Aleo, M. D. 2015. Setting clinical exposure levels of concern for drug-induced liver injury (DILI) using mechanistic in vitro assays. Toxicol. Scil 147, 500-514.
57. Shaw, P., Ditewig, A., Waring, J., Liguori, M., Blomme, E., Ganey, P., Roth, R. 2009a. Coexposure of mice to trovafloxacin and lipopolysaccharide, a model of idiosyncratic hepatotoxicity, results in a unique gene expression profile and interferon gamma-dependent liver injury. Toxicol. Sci. 107, 270-280.
58. Shaw, P., Ganey, P., Roth, R. 2009b. Tumor necrosis factor alpha is a proximal mediator of synergistic hepatotoxicity from rovafloxacin/lipopolysaccharide coexposure. J. Pharmacol. Exp. Ther. 328, 62-68.
59. Stass, H., Dalhoff, A., Kubitza, D., Schuhly, U. 1998. Pharmacokinetics, safety, and tolerability of ascending single doses of moxifloxacin, a new 8-methoxy quinolone, administered to healthy subjects. Antimicrob. Agents Chemother. 42, 2060-2065.
60. Susukida T, Sekine S, Nozaki M, Tokizono M, and Ito K (2015) Prediction of the clinical risk of drug-induced cholestatic liver injury using an in vitro sandwich cultured hepatocyte assay. Drug Metab Dispos 43:1760-1768.
61. Taudorf, S., Krabbe, K., Berg, R., Pedersen, B., Møller, K. 2007. Human models of low-grade inflammation: bolus versus continuous infusion of endotoxin. Clin. Vaccine Immunol. 14, 250-255.
62. Thompson R A, Isin E M, Li Y, Weidolf L, Page K, Wilson I, Swallow S, Middleton B, Stahl S, Foster A J, et al. (2012) In vitro approach to assess the potential for risk of idiosyncratic adverse reactions caused by candidate drugs. Chem Res Toxicol 25: 1616-1632.
63. Uetrecht, J. 1999. New concepts in immunology relevant to idiosyncratic drug reactions: the “danger hypothesis” and innate immune system. Chem. Res. Toxicol. 12, 387-395.
64. Usui T, Mise M, Hashizume T, Yabuki M, and Komuro S (2009) Evaluation of the potential for drug-induced liver injury based on in vitro covalent binding to human liver proteins. Drug Metab Dispos 37:2383-2392.
65. Vanderlinde, R. 1985. Measurement of total lactate dehydrogenase activity. Ann. Clin. Lab. Sci. 15, 13-31.
66. Viechtbauer, W. 2010. Conducting meta-analyses in R with the metafor package. J. of Statistical Software. 36, 1-48.
67. Wang F, Schwarz B T, Graham W V, Wang Y, Su L, Clayburgh D R, Abraham C, and Turner J R (2006) IFN-gamma-induced TNFR2 expression is required for TNFdependent intestinal epithelial barrier dysfunction. Gastroenterology 131: 1153-1163.
68. Ware B R, Berger D R, and Khetani S R (2015) Prediction of drug-induced liver injury in micropatterned co-cultures containing iPSC-derived human hepatocytes. Toxicol Sci 145:252-262.
69. Watkins (2005) Idiosyncratic liver injury: challenges and approaches. Toxicol Pathol 33(1):1-5.
70. Williams, C., Malatjalian, D. 1981. Severe penicillin-induced cholestasis in a 91-year-old woman Dig. Dis. Sci. 26, 470-473.
71. Xu, J., Henstock, P., Dunn, M., Smith, A., Chabot, J., Graff, D. 2008. Cellular Imaging Predictions of Clinical Drug-Induced Liver Injury. Toxicol. Sci. 105, 97-105.
72. Zhang J, Doshi U, Suzuki A, Chang C W, Borlak J, Li A P, and Tong W (2016a) Evaluation of multiple mechanism-based toxicity endpoints in primary cultured human hepatocytes for the identification of drugs with clinical hepatotoxicity: results from 152 marketed drugs with known liver injury profiles. Chem Biol Interact 255:3-11.
73. Zhang J, Salminen A, Yang X, Luo Y, Wu Q, White M, Greenhaw J, Ren L, Bryant M, Salminen W, et al. (2016b) Effects of 31 FDA approved small-molecule kinase inhibitors on isolated rat liver mitochondria. Arch Toxicol 91:2921-2938.
74. Zhu X W, Sedykh A, and Liu S S (2014) Hybrid in silico models for drug-induced liver injury using chemical descriptors and in vitro cell-imaging information. J Appl Toxicol 34:281-288.
75. Zou, W., Beggs, K., Sparkenbaugh, E., Jones, A., Younis, H., Roth, R., and Ganey, P. 2009. Sulindac metabolism and synergy with tumor necrosis factor-alpha in a drug-inflammation interaction model of idiosyncratic liver injury. J. Pharmacol. Exp. Ther. 331, 114-121.

### ABSTRACT

Idiosyncratic, drug-induced liver injury (IDILI) typically affects a small fraction of patients when it occurs, although the effects can be serious and include death. Recent results suggest that tumor necrosis factor alpha (TNF) interacts with drugs that cause IDILI to kill hepatocytes. Using a set of 24 drugs, we demonstrated previously that cytotoxicity in the presence and absence of TNF can be used to classify with excellent accuracy the propensity of drugs to cause IDILI (Maiuri et al., JPET 362: 459, 2017). The statistical modeling for that classification relied on covariates derived from complete concentration response relationships (CRRs). However, sometimes complete CRRs cannot be determined (e.g., due to limited drug solubility), which would limit the usefulness of this approach in preclinical evaluation of drug candidates. We hypothesized that successful classification could be accomplished using only data from the low end of the CRRs. We tested this hypothesis using the same data set as previously modeled; however, concentration-response data were censored above the EC50 to conduct CRR modeling using only the low-dose region. Multiple curve-fitting models were compared for their ability to fit the low-dose region for each CRR. Akaike Information Criteria (AIC) were used to select an optimal fitting model for each drug to be used to estimate a point of departure (POD) for the CRR. PODs were estimated for each drug in the absence and presence of TNF coexposure (covariates 1 and 2). These PODs, the difference between PODs (covariate 3), and the ratio of PODs for vehicle and TNF-treated cells (covariate 4) were used to create statistical classification models. Performance of the models was evaluated using receiver operating characteristic (ROC) analysis. Individually, covariates 1 through 4 yielded areas under the ROC curves (AUCs) of 0.72, 0.79, 0.83, and 0.83, respectively. The classification model incorporating both PODs (i.e., covariates 1 and 2) classified drugs with high selectivity and specificity and an AUC of 0.96. These results suggest that models employing covariates comprising PODs estimated from incomplete CRRs can accurately classify drugs according to their IDILI potential. (Supported by NIH grant R01 DK112695 and NIH training grant 5T32ES7255-29)

### Introduction

There exists a need for assays that can predict, during drug development, which drug candidates are likely to cause idiosyncratic, drug-induced liver injury (IDILI). Using a set of 24 drugs, we demonstrated previously that cytotoxicity of HepG2 cells in the presence and absence of tumor necrosis factor alpha (TNF) can be used to classify drugs according to their IDILI liability (Maiuri et al., JPET 362: 459, 2017). The statistical modeling for that classification relied on covariates (eg, EC50s, maximal responses) derived from complete concentration response relationships (CRRs). Sometimes, complete CRRs cannot be determined (e.g., due to limited drug solubility). Accordingly, we tested the hypothesis that successful IDILI classification could be accomplished using incomplete CRRs. In this study, we used the lower end of the concentration-response curves (CRCs) for the set of 24 drugs employed in our earlier study to simulate cases for which complete CRRs are not achievable. The incomplete CRRs were then used to estimate a point of departure (POD) for each drug in the presence and absence of TNF for use as covariates in constructing a best-fit statistical classification model using logistic regression.

### Statistical Methods and Results

EC50s (drug concentrations corresponding to 50% lactate dehydrogenase (LDH) release as a measure of cell death) were estimated using a 4-parameter log-logistic model of complete CRRs for each of 24 drugs. Partial, or censored, CRRs were then created using concentrations at or below the EC50.

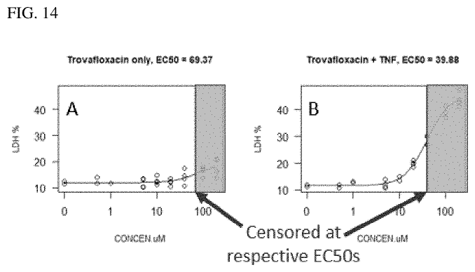

FIG. 14: Graphs of complete CRRs (shown as points) for trovafloxacin alone (A) or in the presence of TNF (B) and fitted 4-parameter log-logistic CRC models (shown as lines). For this study, CRRs were censored above their respective EC50s to simulate incomplete CRRs.

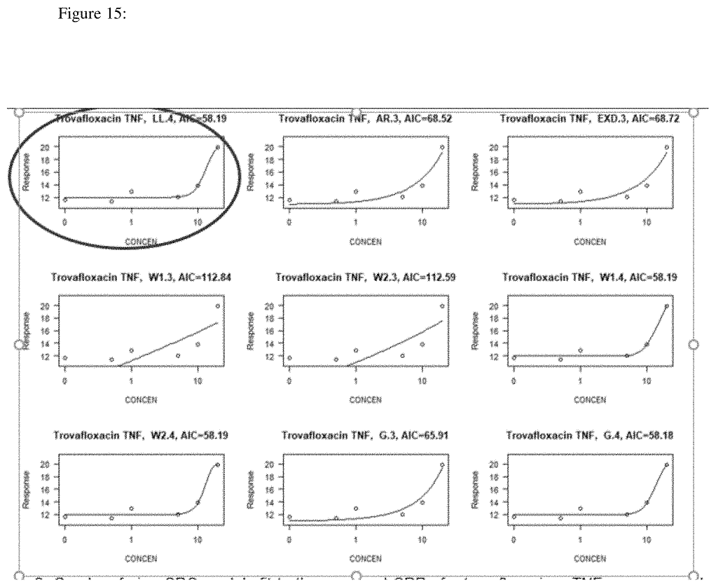

Nine CRC models were fit to the partial CRRs for all 24 drugs and compared to identify the best-fitting CRC model overall. Models were compared using Akaike Information Criteria (AIC; Akaike 1973, Burnham & Anderson 2002). The 4-parameter log-logistic model was selected to model censored CRRs for each of the 24 drugs since it provided the best fit (lowest AIC) for the majority of drugs. FIG. 15 illustrates the model comparison for trovafloxacin+TNF.

FIG. 15: Graphs of nine CRC models fit to the censored CRRs for trovafloxacin+TNF as an example. AIC values, measures of model fit, are shown for each model. Lower AIC values indicate better fit. The CRC model selected for estimation of covariates is circled in red. LL.4=4-parameter log-logistic model, AR.3=3-parameter asymptotic regression model, EXD.3=3-parameter exponential model, W1.3&W2.3=3-parameter Weibull models, W1.4&W2.4=4-parameter Weibull models, G.3=3-parameter Gompertz model, G.4=4-parameter Gompertz model.

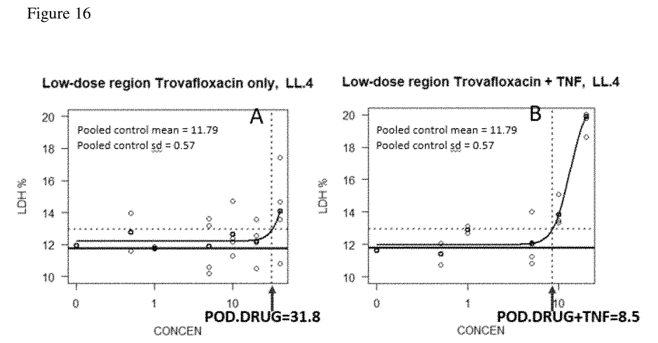

Points of Departure (PODs) were calculated for each drug alone and in the presence of TNF as shown in FIG. 16 using 4-parameter log-logistic CRCs. PODs provided a robust measure to characterize each of the CRCs using the low-concentration regions of the CRRs. In addition to POD.DRUG and POD.DRUG+TNF, POD.DIFF (i.e., POD.DRUG−POD.DRUG+TNF) and POD.RATIO (i.e., POD.DRUG/POD.DRUG+TNF) were used as covariates in the logistic regression model developed to classify the drugs according to their IDILI status.

FIG. 16: 4-parameter log-logistic CRC models fit to the censored CRRs (low-concentration region) for trovafloxacin alone (A) and in the presence of TNF (B). The horizontal blue line illustrates the mean control value estimated by pooling LDH % responses at the zero concentrations for both CRRs above. The horizontal dashed red line illustrates an upper limit of the pooled control data as the control mean plus 2 control standard deviations. PODs were then calculated for each curve as the effective concentration corresponding to the upper limit of the pooled control data, shown as vertical dashed red lines. Note that POD.DRUG occurs at a relatively higher concentration than POD.DRUG+TNF for trovafloxacin.

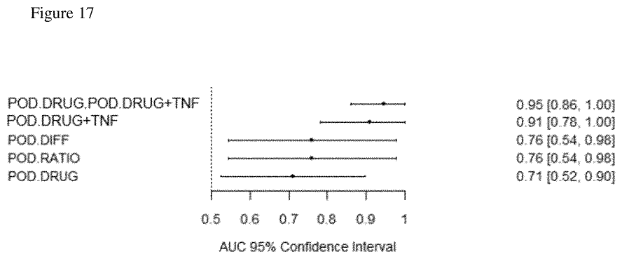

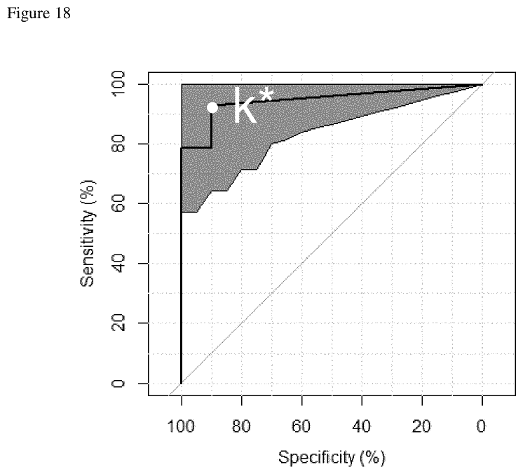

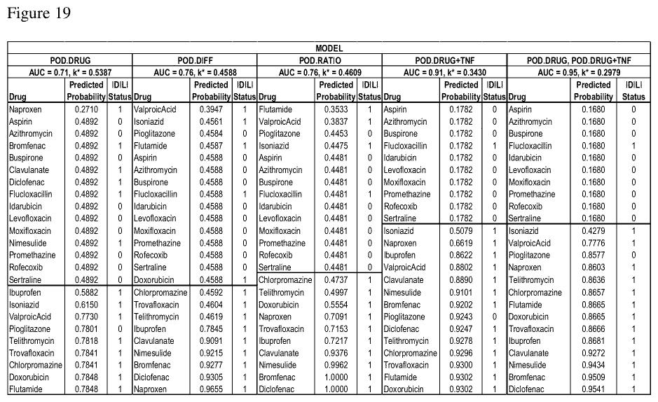

A dataset of the four covariates listed above and IDILI status for the 24 drugs was compiled for use in classification model development. Five logistic regression models were evaluated: one model with each covariate alone and a model using both POD.DRUG and POD.DRUG+TNF. Receiver operating characteristic (ROC) curves were developed for each model to evaluate classification accuracy, represented by the area under the curve (AUC). FIG. 17 provides a comparison of the classification accuracy for each model. The model including both POD.DRUG and POD.DRUG+TNF as covariates yielded the best classification accuracy with an AUC of 0.95. FIG. 18 illustrates the ROC curve for this model. Drug classifications according to the various models are compared in FIG. 19.

FIG. 17: Whisker plots comparing classification model performance The model with covariates POD.DRUG and POD.DRUG+TNF has the highest AUC of 0.95 and the narrowest 95% confidence interval, ranging from 0.86 to 1.00. A model with perfect classification would yield an AUC of 1.0. A model with no classification ability (i.e., a model that classifies no better than random chance) would have an AUC of 0.5.

FIG. 18 ROC Curve for the best classification model. AUC=0.95. The blue shaded area illustrates the confidence limits around the sensitivity estimate for the full range of possible specificities. At the optimal cutoff, k*, sensitivity and specificity are at their highest combined values; the specificity of the classification model at k* is 90.0% and the sensitivity is 92.9%. Other cutoffs (k's) can be used to represent a variety of risk tolerances (i.e., sensitivity versus specificity).

FIG. 19: Predicted probability of drug being IDILI+ for each drug according to the five models. Models are sorted left to right from worst to best. AUCs and optimal cutoffs (k*) are shown for each model. Within each model, drugs are sorted top to bottom from lowest to highest predicted probabilities. A dark line divides the probabilities by classification using the k* cutoff for each model. Models for POD.DRUG, POD.DIFF, and POD.RATIO have lower AUCs and demonstrate poorer ability to separate between IDILI+ and − drugs (i.e., the difference between probabilities for + and − drugs is small). The other two models (i.e., POD.DRUG+TNF and POD.DRUG with POD.DRUG+TNF) provide a wider gap in probabilities between the IDILI+ and − drugs. This is a desirable characteristic in addition to high AUC values, high sensitivity, and high specificity.

### Conclusions

In this study, we tested the hypothesis that successful IDILI classification could be accomplished using incomplete CRRs. Utilizing the same drug set as our initial study (Mairuri et al., 2017), but with concentration-response curves artificially censored above their respective EC50s, we retained the ability to classify drugs accurately according to IDILI liability.

### REFERENCES

Akaike, H. (1973), “Information theory and an extension of the maximum likelihood principle”, in Petrov, B. N.; Csáki, F., 2nd International Symposium on Information Theory, Tsahkadsor, Armenia, USSR, Sep. 2-8, 1971, Budapest: Akadémiai Kiadó, pp. 267-281.
Burnham, K. P.; Anderson, D. R. (2002), Model Selection and Multimodel Inference: A practical information-theoretic approach (2nd ed.), Springer-Verlag, ISBN 0-387-95364-7.
Maiuri, A. R., B. Wassink, J. D. Turkus, A. B. Breier, T. Lansdell, G. Kaur, S. L. Hession, P. E. Ganey and R. A. Roth (2017). “Synergistic Cytotoxicity from Drugs and Cytokines In Vitro as an Approach to Classify Drugs According to Their Potential to Cause Idiosyncratic Hepatotoxicity: A Proof-of-Concept Study.” J Pharmacol Exp Ther 362(3): 459-473.
Xavier Robin, Natacha Turck, Alexandre Hainard, Natalia Tiberti, Frederique Lisacek, Jean-Charles Sanchez and Markus Müller (2011). pROC: an open-source package for R and S+ to analyze and compare ROC curves. BMC Bioinformatics, 12, p. 77. DOI: 10.1186/1471-2105-12-77.

### INCORPORATION BY REFERENCE

All publications, including but not limited to patents and patent applications, cited in this specification, to the extent that they provide exemplary procedural or other details supplementary to those set forth herein, are specifically incorporated herein by reference as if each individual publication were specifically and individually indicated to be incorporated by reference herein as though fully set forth.

### EQUIVALENTS

Those skilled in the art will recognize, or be able to ascertain using no more than routine experimentation, many equivalents of the specific embodiments of the invention described herein. Such equivalents are intended to be encompassed by the following claims.

1. An in vitro method of classifying a drug according to the drug's potential to cause liver cell injury, comprising:
a) obtaining a population of liver cells;
b) contacting the population of liver cells with a drug provided at a range of concentrations;
c) contacting the population of liver cells with a cytokine provided at a range of concentrations;
d) determining cytotoxicity of the population of liver cells;
e) generating a concentration-response curve;
f) defining covariates from the curve using a four-parameter logistic model;
g) developing a classification model using logistic regression of covariates defined in step h) to generate a logistic regression model; and
i) evaluating by receiver operating characteristic (ROC) the optimal classification model and covariate combination, to thereby classify the drug according to the drug's potential to cause liver injury.

2. The method of claim 1, wherein the liver cells are primary human heptocytes.

3. The method of claim 1, wherein the hepatoma cells are human hepatoma cells.

4. The method of claim 1, wherein the human hepatoma cells are HepG2.

5. The method of claim 1, wherein the liver cell injury is a hepatocellular injury.

6. The method of claim 1, wherein the liver cell injury is liver death.

7. The method of claim 5, wherein the hepatocellular injury is idiosyncratic, drug-induced liver injury (IDILI).

8. The method of claim 1, wherein the drug is selected from the group consisting of steroidal or nonsteroidal anti-inflammatory drugs (NSAIDs), antibiotic, anti-viral, anti-bacterial, anti-fungal, chemotherapeutic, small molecule drugs of any pharmacologic class, cardiac, pulmonary, lipid-modulating, neuromodulatory, analgesic, drugs that modify blood coagulation, gastrointestinal (GI) drugs, anti-convulsants, and endocrine drugs.

9. The method of claim 8, wherein the drug is selected from any one of the drugs set forth in Table 1.

10. The method of claim 1, wherein the cytokine is selected from the group consisting of IL-3, IL-4, tumor necrosis factor-alpha (TNF-α), TNF-β, LT-β, interleukin-2 (IL-2), IL-7, IL-9, IL-15, IL-13, IL-5, IL-1α, IL-1β, interferon-gamma (IFN-γ), IL-10, IL-17, IL-16, IL-18, HGF, IL-11, MSP, FasL, TRAIL, TRANCE, TWEAK, CD27L, CD30L, CD40L, APRIL, TALL-1, 4-1BBL, OX40L, GITRL, IGF-1, IGF-II, MSP, FGF-α, FGF-β, FGF-3-19, NGF, BDNF, NTs, Tpo, Epo, Ang1-4, PDGF-AA, PDGF-BB, VEGF-A, VEGF-B, VEGF-C, VEGF-D, PIGF, EGF, TGF-α, AR, BTC, HRGs, HG-EGF, SMDF, OB, CT-1, CNTF, OSM, MK, and PTN.

11. The method of claim 10, wherein the cytokine is TNF-α, IFN-γ, or both.

12. The method of claim 1, wherein the cytotoxicity is measured as lactate dehydrogenase (LDH) activity released from cells.

13. The method of claim 1, wherein the cytotoxicity is measured as percent (LDH) activity released from cells.

14. The method of claim 1, wherein the cytotoxicity is measured as percent cellular ATP released from cells.
